(19) United States
(12) Patent Application Publication
(10) Pub. No.: US 2025/0134826 A1
(43) Pub. Date: May 1, 2025

(54) NITRIC OXIDE RELEASING FILMS AND SYSTEMS

(71) Applicants: NOTA Laboratories, Inc., Ann Arbor, MI (US); Niterra North America, Inc., Wixom, MI (US); Niterra Co., Ltd., Nagoya (JP)

(72) Inventors: Andrew P. Hunt, Saline, MI (US); Malcolm R. Kahn, Franklin Lakes, NJ (US); Glenn B. Martin, Redford Twp., MI (US); Corey J. White, Ann Arbor, MI (US); Alexander K. Wolf, Ann Arbor, MI (US); Kyosuke Nakamura, Wixom, MI (US); Hiroshi Kasakawa, Nagoya (JP)

(21) Appl. No.: 18/821,209

(22) Filed: Aug. 30, 2024

Related U.S. Application Data
(60) Provisional application No. 63/594,380, filed on Oct. 30, 2023.

(51) Int. Cl. A61K 9/70; A61K 33/00; B01J 19/12; C01B 21/24

(52) U.S. Cl. CPC A61K 9/7007; A61K 33/00; B01J 19/122; C01B 21/24; B01J 2219/0869; B01J 2219/0879; B01J 2219/1203

(57) ABSTRACT

An example of a nitric oxide (NO) releasing film includes a substrate; an NO donor film attached to the substrate; and an NO permeable and light transparent film positioned on the NO donor film. The NO donor film includes a polymer matrix selected from the group consisting of a non-UV curable polyurethane, polyvinyl butyral, polystyrene, copolymers of styrene, block copolymers of styrene, poly(ethersulfone), polyvinylpyrrolidone, polyvinyl acetate, poly(ethylene-co-vinylacetate), and combinations thereof; and solid, light sensitive NO donor particles distributed throughout the polymer matrix. The solid, light sensitive NO donor particles have a volume-weighted mean diameter of 50 μm or less. In one example, the NO donor film further includes an additive, such as NO₂ scrubber particles, a radical stabilizer, dispersant, and/or an anti-skinning additive.

### CROSS-REFERENCE TO RELATED APPLICATIONS

This application claims the benefit of U.S. Provisional Application Ser. No. 63/594,380, filed Oct. 30, 2023, the content of which is incorporated by reference herein in its entirety.

### STATEMENT REGARDING FEDERALLY SPONSORED RESEARCH OR DEVELOPMENT

This invention was made with government support under 1 R43 HL149556-01 and 1 R44 HL149556-02A1, each of which was awarded by the National Institutes of Health (NIH). The government has certain rights in the invention.

### BACKGROUND

Nitric oxide (NO) is an endogenous gas molecule that has been shown to have several important physiological functions, examples of which include its unique vasodilating properties, wound healing properties, angiogenesis promoting properties, cancer-fighting potency, anti-platelet activity, and anti-microbial/anti-viral activity. NO has been used for infection, inflammation, and fibrosis control/minimization, biofilm formation prevention, and inhalation therapy.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a schematic, cross-sectional view of one example of a nitric oxide (NO) releasing film disclosed herein;

FIG. 1B is a schematic, cross-sectional view of another example of a nitric oxide (NO) releasing film disclosed herein;

FIG. 2 is a schematic, perspective view of an example of a nitric oxide (NO) releasing system disclosed herein;

FIG. 3 is a schematic, perspective view of an example of a nitric oxide gas delivery and monitoring system disclosed herein;

FIG. 4 is a graph depicting the amount of nitric oxide (NO, part per million (ppm), left Y axis) and the amount of nitrogen dioxide (NO₂, ppm, right Y axis) over time (minutes, X axis) generated during a target setpoint experiment using one example of the nitric oxide (NO) donor film disclosed herein;

FIG. 5 is a graph depicting the amount of nitric oxide (NO, part per million (ppm), left Y axis) and the amount of nitrogen dioxide (NO₂, ppm, right Y axis) over time (minutes, X axis) generated during a target setpoint experiment using another example of the NO donor film disclosed herein;

FIG. 6 is a graph depicting the amount of nitric oxide (NO, part per million (ppm), left Y axis) and the amount of nitrogen dioxide (NO₂, ppm, right Y axis) over time (minutes, X axis) generated during a target setpoint experiment using still another example of the NO donor film disclosed herein;

FIG. 7 is a graph depicting the amount of nitric oxide (NO, part per million (ppm), left Y axis) and the amount of nitrogen dioxide (NO₂, ppm, right Y axis) generated over time (minutes, X axis) during a depletion experiment using yet another example of the NO donor film disclosed herein;

FIG. 8 is a graph depicting the amount of nitric oxide (NO, part per million (ppm), left Y axis) over time (minutes, X axis) for example NO releasing films exposed to different wavelengths of light;

FIG. 9 is a graph depicting the amount of nitric oxide (NO, part per million (ppm), left Y axis) over time (minutes, X axis) for different example NO releasing films containing NO donor particles of different particle sizes;

FIG. 10 is a schematic and perspective view of an example of a portable nitric oxide releasing device that can receive two cartridges that contain the NO releasing film described herein;

FIG. 11 is a schematic and perspective view of the interior of the portable nitric oxide releasing device of FIG. 10;

FIG. 12 is a schematic and perspective view of some of the components that are contained within the interior of the portable nitric oxide releasing device of FIG. 10;

FIG. 13 is a schematic and perspective view of one of the cartridges and the NO releasing film of the portable nitric oxide releasing device of FIG. 10, where the arrows depict an example of the nitric oxide generation and flow;

FIG. 14 is a schematic and perspective view of a portable system including the portable nitric oxide releasing device of FIG. 18 and a flow diagram depicting its use in a patient care setting;

FIG. 15A and FIG. 15B are, respectively, perspective and side views of another example of a cartridge that can house the NO releasing film disclosed herein;

FIG. 16A and FIG. 16B are, respectively, perspective and side views of yet another example of a cartridge that can house the NO releasing film disclosed herein;

FIG. 17A and FIG. 17B are each perspective views of still another example of a cartridge that can house the NO releasing film disclosed herein;

FIG. 18 is a schematic and perspective view of another example of a portable nitric oxide generating device that can receive two cartridges that contain the NO generating film described herein;

FIG. 19 is a schematic and perspective view of some of the components that are contained within the interior of the portable nitric oxide generating device of FIG. 18;

FIG. 20 is a perspective view of another example of a cartridge that can house the NO generating film disclosed herein;

FIG. 21 is a bottom view of the cartridge of FIG. 20;

FIG. 22 is a perspective view of an interior housing of the cartridge of FIG. 21 with a cap and a lid in place;

FIG. 23 is a perspective view of the interior housing of the cartridge of FIG. 20 and the components contained therein;

FIG. 24 is a schematic and perspective view of the cartridge of FIG. 20, depicting both the exterior and the interior;

FIG. 25 is a graph depicting the amount of nitric oxide (NO, part per million (ppm), left Y axis) and the amount of nitrogen dioxide (NO₂, ppm, right Y axis) generated over time (minutes, X axis) during a depletion experiment using yet another example of the NO donor film disclosed herein; and

FIG. 26 is a graph depicting the amount of nitric oxide (NO, part per million (ppm), left Y axis) and the amount of nitrogen dioxide (NO₂, ppm, right Y axis) over time (minutes, X axis) generated during a target setpoint experiment using still another example of the NO donor film disclosed herein.

### DETAILED DESCRIPTION

Inhaled nitric oxide may be useful in a variety of applications, including substance delivery (e.g., inhaled antiseptic agent), treatment plans (e.g., for lung failure or pulmonary hypertension), and invasive medical procedures (e.g., during and after organ transplants, such as lung, heart and kidney transplants). Inhaled NO has been effective in enhancing pulmonary vasodilation, lowering pulmonary vascular resistance, and treating neonates that suffer from hypoxic respiratory failure.

Disclosed herein is an NO releasing film that produces therapeutic and higher levels of NO at a constant rate, due, at least in part, to the inclusion of solid, light sensitive NO donor particles having a diameter of 50 μm or less. The relatively small particles provide a greater surface area per unit mass (compared to larger particles), and thus provide an increased area for light exposure (which triggers NO release).

As used herein, the terms “nitric oxide releasing film” and “NO releasing film” may refer to a single film that is capable of releasing nitric oxide upon exposure to a predetermined wavelength of light, or to a film stack that includes at least one film that is capable of releasing nitric oxide upon exposure to a predetermined wavelength of light. The single film may also be referred to herein as a “nitric oxide donor film” or an “NO donor film.”

The membrane(s)/layer(s) of the NO releasing films disclosed herein are also flexible, which enables them to be coiled and/or spooled. The spooled film can be incorporated into a relatively small device that is able to continuously deliver relatively high ppmv NO doses. A “relatively high ppmv” NO dose refers to NO delivery ranging from 81 ppmv to 400 ppmv. A “moderate ppmv” NO dose refers to NO delivery ranging from 11 ppmv up to, but not including, 81 ppmv. In one example, the NO delivery ranges from 40 ppmv to 60 ppmv (of NO) at a gas flow of 8 L/min. A “low ppmv” NO dose refers to NO delivery ranging from 0.05 ppmv up to, but not including, 11 ppmv. In all NO delivery ranges, gas flow rates may range from 0.05 L/min to 100 L/min. In some examples, the relatively high NO dose may be achieved using single or double sided illumination of either a single sided or double sided film. Lower doses are most easily achieved using the single sided film disclosed herein and single sided illumination. For sustained delivery at these doses, the film is advanced as described herein in connection with the method.

Single sided illumination can be used to produce NO from the film, however double sided illumination of the single sided film may generate a higher dose for a shorter time period than if single sided illumination were used. Both single and double sided illumination may be used to compensate for older films where some unintended NO release may have taken place. Single sided illumination is satisfactory for NO production, however double sided illumination may also drive off NO in a given section of the film more quickly than if single sided illumination were used. Due to the greater efficiency of NO release, faster film advancement speeds are obtainable for double sided illumination and NO delivery, and thus the cartridge may be expended sooner than if single sided illumination were used.

In some of the examples described herein, the NO releasing film includes a substrate and an NO donor film attached to the substrate. As used herein, the term “attached” refers to the state of two things being joined, fastened, adhered, connected or bound to each other, either indirectly or directly. As an example of indirect attachment, a base binding layer may be positioned between the substrate and the NO donor film. As an example of direct attachment, the NO donor film may be in contact with a surface of the substrate, without any intervening layer(s).

### NO Releasing Film

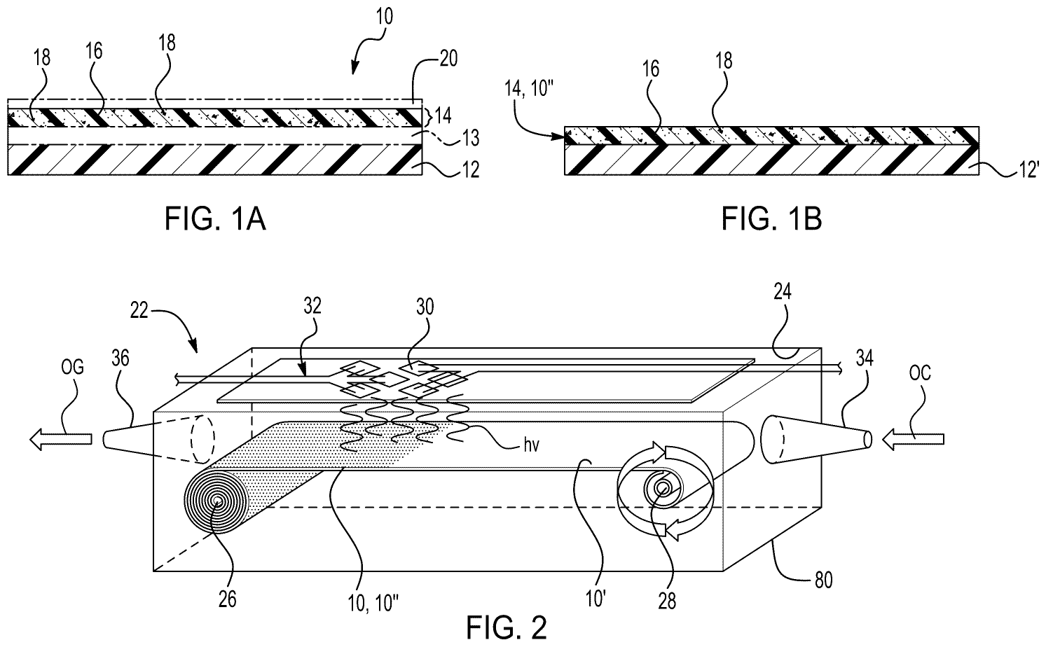

As mentioned, the NO releasing film disclosed herein may be a multi-layered structure (e.g., see reference numeral 10 in FIG. 1A) or a single layered structure (see reference numeral 10″ in FIG. 1B).

The NO releasing film 10″ shown in FIG. 1B includes a polymer matrix 16 selected from the group consisting of non-ultraviolet (UV) curable polyurethane, polyvinyl butyral, polystyrene, copolymers of styrene, block copolymers of styrene, poly(ethersulfone), polyvinylpyrrolidone, polyvinyl acetate, poly(ethylene-co-vinylacetate), and combinations thereof, and solid, light sensitive NO donor particles 18 distributed throughout the polymer matrix 16, wherein a volume-weighted mean diameter of the solid, light sensitive NO donor particles 18 is 50 μm or less. In FIG. 1B, the NO releasing film 10″ is shown on a temporary substrate 12′, which will be discussed further in reference to the methods. In some examples, the NO releasing film 10″ consists of the polymer matrix 16 and the solid, light sensitive NO donor particles 18 having a diameter of 50 μm or less.

In the NO releasing film 10 shown in FIG. 1A, the polymer matrix 16 and the solid, light sensitive NO donor particles 18 distributed throughout the polymer matrix 16 form an NO donor film 14; and the NO releasing film 10 further comprises a substrate 12 attached to the NO donor film 14. As shown in FIG. 1A, the NO releasing film 10 may also include a base binding layer 13 positioned between the substrate 12 and the NO donor film 14 and/or an NO permeable and light transparent film 20 positioned on the NO donor film 14. Each of these components will be further described herein in reference to FIG. 1A. In some examples, the NO donor film 14 consists of the polymer matrix 16 and the solid, light sensitive NO donor particles 18 having a diameter of 50 μm or less.

The substrate 12 may be any porous or non-porous material formed of a polymer that exhibits low oxygen (O₂) permeability/solubility.

Non-porous substrates are materials with non-perforated surfaces that restrict the diffusion of both liquids and gases. If the non-porous substrate has any pores, the size of such pores is ≤1 nm. The non-porous substrate surfaces may be flat or contoured.

Porous substrates include pores or voids that allow the diffusion of liquids and/or gases of a particular size. In general, the pores may have a size ranging from the nanoscale (having a size ranging from about 2 nm to about 50 nm) to the macroscale (having a size ranging from about 100 nm to about 10 μm). In some examples, the pores of the substrate 12 are microporous, i.e., within the range from about 50 nm to about 100 nm. In other examples, the pores of the substrate 12 are about 0.4 μm or about 1.4 μm.

By “low oxygen permeability/solubility” and “low O₂ permeability/solubility,” it is meant that the permeability of the polymer used to make the substrate 12 (or polymer matrix 16) is 10*10⁹ cm³ (RTP)*cm/s*cm²*cmHg or less. In one example, the polymer is high-density polyethylene (HDPE) having an O₂ permeability of 0.1*10⁹ cm³ (RTP)*cm/s*cm²*cmHg. In another example, the polymer is PET having an O₂ permeability of 0.0019*10⁹ cm³ (RTP)*cm/s*cm²*cmHg. It is desirable for the polymer (and thus the substrate 12) to take up as little oxygen (O₂) as possible, as O₂ reacts with nitric oxide (NO) to generate undesirable nitrogen dioxide (NO₂) byproduct. When the polymer that forms the substrate 12 is devoid of oxygen gas, the NO releasing film 10 can effectively photolytically release NO without also releasing appreciable levels of NO₂. As such, it is desired that neither oxygen gas nor NO permeate into the substrate 12, which reduces the time that the NO is in contact with O₂ prior to its release from the NO releasing film 10 into the gas phase.

The substrate 12 is also flexible. By “flexible,” it is meant that the substrate 12 is able to be coiled and spooled without breaking or cracking. Quantitatively, the substrate 12 can exceed 20,000 folding cycles when tested on an MIT flex tester (TAPPI method T-423).

In some examples disclosed herein, the substrate 12 is polyethylene terephthalate (PET) or a variant thereof (e.g., matte PET). The PET may be untreated or treated with a corona treatment, depending upon the polymeric matrix 16 that is being used. In one example, the PET is an extruded sheet having pores less than 100 nm. In other examples, the PET may also be electrospun with a larger pore size. PET may have a glossy surface finish, which is transparent and smooth. A variant of PET is matte PET, which is a biaxially oriented polyester film. The biaxially oriented polyester film has a medium haze and grainy surface, giving it semi-transparent optical qualities and rougher surface areas. The rougher surface areas may promote improved adhesion of the polymer matrix 16 or a base binding layer 13 to the NO donor film 14. Other PET variants may include a metallized coating, such as aluminum.

In other examples disclosed herein, the substrate 12 is made up of flash-spun or electrospun non-woven materials. Flash-spun or electrospun non-woven materials provide the substrate 12 with a webbed, fibrous surface structure. Any flash-spun or electrospun non-woven material may be used, as long as it is flexible as defined herein. Some examples include flash-spun high-density polyethylene (HDPE) fibers, electrospun polyvinylidene fluoride (PVDF), PVDF membranes, polytetrafluoroethylene (PTFE), polypropylene (PP), PELLON® (80/20 cotton/polyester blend textile), glass fibers (GF), filter membranes, or nylon (polyamide) screen materials. It is believed that other substrate 12 materials, such as mixed cellulose ester (MCE) and benzoin methyl ether (BME), may be used, as long as the solvent mixture used during deposition of the polymer matrix 16 dissolves the polymer matrix 16 without deleteriously affecting the MCE or the BME. One specific example of a suitable substrate 12 material includes high-density polyethylene (HDPE) fibers, such as TYVEK® (a non-woven material made up of synthetic flash-spun high-density polyethylene fibers from DuPont, e.g., medical grade products with a thickness ranging from about 50 μm to about 254 μm (from about 2.0 to about 10.0 mil), such as TYVEK® 1073B and TYVEK® 1059B). In some examples, the substrate 12 material includes a non-woven material made up of synthetic flash-spun high-density polyethylene having a thickness ranging from about 150 μm to about 204 μm (from about 6.2 mil to about 7.8 mil), or from about 101 μm to about 191 μm (from 6.5 mil about to about 7.5 mil). In general, the thickness of the substrate 12 may range from about 10 μm to about 2540 μm (from about 0 mil to about 100 mil), which may depend upon the material used and the desire to coil/spool the substrate 12. In one example, the thickness is 508 μm or less (20 mil or less).

In some examples, the NO donor film 14 is attached directly to the substrate 12. The porous, and in some instances fibrous, structure of the substrate 12 provides several surfaces for the NO donor film 14 to anchor to during immobilization (e.g., solvent evaporation). In some examples, the non-porous or porous substrate may be exposed to a corona treatment in order to improve the adhesion between the substrate and the NO donor film 14. Corona treatment increases the surface energy of plastic films to increase wettability and adhesion of inks, coatings and adhesives. It is believed that at least some of the NO donor film 14 may be physically restrained within at least some of the pores of the porous substrate 12 examples, which results in stronger binding than surface adsorption alone. For example, films bound to porous, non-fibrous substrates via surface adsorption, without being restrained or adhered with an additional adhesive material, may delaminate, crack, and peel off from the underlying substrate, especially when coiled or spooled. These examples may exhibit overall flexibility and may resist sticking when coiled.

In other examples, the NO donor film 14 is indirectly attached to the substrate 12 through the base binding layer 13 (shown in phantom in FIG. 1A). The base binding layer 13 may be an adhesive or primer layer that promotes the attachment of the polymer matrix 16 and the solid, light sensitive NO donor particles 18 to the substrate 12, and that also reduces the potential for or prevents delamination. It is to be understood that any suitable adhesive may be used, so long as it does not compromise the flexibility of the NO releasing film 10. The base binding layer 13 may be composed of the same polymer that is included as the polymer matrix 16 or may be a different polymer than the polymer matrix 16, and does not include the solid, light sensitive NO donor particles 18. In one example, the base binding layer 13 is polyvinyl butyral (e.g., BM-SZ from SEKISUI Chemical Co.).

The NO donor film 14 of FIG. 1A and the single layer NO generating film 10″ of FIG. 1B includes the polymer matrix 16 and the solid, light sensitive NO donor particles 18 distributed throughout the polymer matrix 16. Each of the films 14, 10″ is a solid state storage film.

The polymer matrix 16 exhibits low O₂ permeability/solubility, as it is defined herein.

The polymer matrix 16 is selected from the group consisting of non-UV curable polyurethane, polyvinyl butyral, polystyrene, copolymers of styrene, block copolymers of styrene, poly(ethersulfone), polyvinylpyrrolidone, polyvinyl acetate, poly(ethylene-co-vinylacetate), and combinations. Other polymer materials that exhibit the low O₂ permeability/solubility and flexibility may also be suitable.

When the polymer matrix 16 is a non-UV curable polyurethane, the backbone of the polymer matrix 16 includes urethane linkages formed between diisocyanate monomers and isocyanate reactive groups, such as hydroxyls (e.g., as part of a diol or other polyol). Chain extenders and/or capping agents can also be used, respectively, to extend the polyurethane chain and terminate chain extension.

Suitable diisocyanate monomers include hexamethylene-1,6-diisocyanate (HDI), 2,2,4-trimethyl-hexamethylene-diisocyanate (TDMI), 1,12-dodecane diisocyanate, 2,4,4-trimethyl-hexamethylene diisocyanate, 2-methyl-1,5-pentamethylene diisocyanate, isophorone diisocyanate (IPDI), methylene diphenyl diisocyanate (MDI), or 1-Isocyanato-4-[(4-isocyanatocyclohexyl)methyl]cyclohexane) (H12MDI, i.e., 4,4′-Methylenedicyclohexyl diisocyanate). Suitable diols or polyols include pentyl glycols (e.g., neopentyl glycol); C₄ to C₁₀ alkyldiol (e.g., 1,4-butanediol, hexane-1,6-diol); C₄ to C₁₀ alkyl dicarboxylic acids (e.g., adipic acid); and aromatic dicarboxylic acids, e.g., phthalic acid. The polyurethane is a non-UV curable polyurethane, meaning that it does not include UV curable functional groups, such as acrylate groups.

The polyurethane example of the polymer matrix 16 may include both soft and hard segments, where the soft segments are composed of a polyether or polyester polyol and the hard segments are composed of diisocyanate and, when used, the chain extender. In an example, the ratio of soft segments to hard segments ranges from about 65:35 (13:7) to about 60:40 (3:2). The ratio of soft segments to hard segments may be adjusted so that the polyurethane polymer matrix 16 has a Shore hardness value ranging from about 35A to about 80D.

In one example, the polyurethane example of the polymer matrix 16 is an aliphatic polyether-based thermoplastic polyurethane having a Shore A hardness value of 72 (measured using a Shore durometer tool) and a flexural modulus (psi) of 1,000. An example of this polyurethane is commercially available under the tradename TECOFLEX™ TPU (from Lubrizol Corp.). The backbone of this particular polyurethane is shown below:

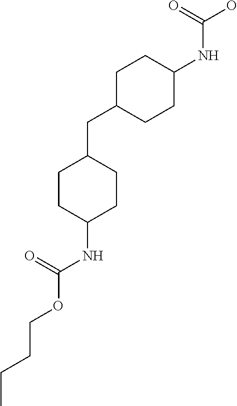 [SMILES: CCCCOC(=O)NC1CCC(CC2CCC(NC(=O)O)CC2)CC1]

Polyvinyl butyral is another suitable polymer matrix 16 material. Polyvinyl butyral consists of three monomeric subunits—vinyl butyral, vinyl alcohol, and vinyl acetate, each of which is shown below:

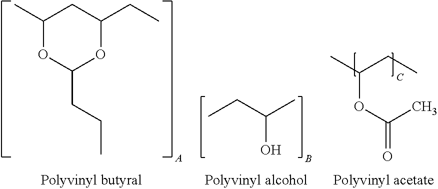

In an example, the polyvinyl butyral has a hardness ranging from about 18D to about 60D. In one specific example, the polyvinyl butyral has the following properties: average molecular weight ranging from about 1,000 to about 15,000; viscosity from about 30 to about 300 mPa-s in a 5 wt % to 10 wt % solution; from 16 wt % to 25 wt % alcohol content, 19 wt % or less acetate content, and from about 65 wt % to about 83 wt % of acetal content; and a glass transition temperature ranging from about 65° C. to about 115° C. Any of the polyvinyl butyral formulations commercially available from multiple manufactures, example: Sekisui Co. (whose formulations may have different percentages of the repeating unit) may be used, as long as they exhibit the properties set forth herein for the polymer matrix 16.

Still another suitable polymer matrix 16 is polystyrene or styrene copolymers or styrene block copolymers. As shown below, examples of styrene block copolymers include units of styrene and isoprene in blocks or units of styrene and butadiene in blocks.

Polystyrene has the structure:

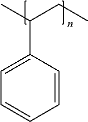

and is made up of polymerized styrene monomers. In an example, the polystyrene has a hardness ranging from about 90A to about 90D. In one specific example, the average molecular weight of the polystyrene ranges from about 10,000 to about 600,000 and the glass transition temperature ranges from about 90° C. to about 212° C.

Polystyrene-block-polybutadiene-block-polystyrene is one example of a styrene block copolymer:

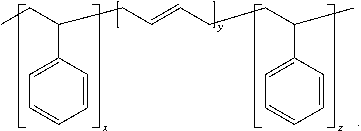

In one example, the hardness of this block copolymer ranges from about 60D to about 80D based on the content of butadiene in the block copolymer. In one specific example, the polystyrene-block-polybutadiene-block-polystyrene has the following properties: a hardness ranging from about 40 to about 80 (Shore A); and from about 20 wt % to about 40 wt % styrene content and from about 60 wt % to about 80 wt % butadiene content. It is to be understood that other percentages of the monomers may be used, and that a higher butadiene content results in a softer copolymer or block copolymer.

Polystyrene-block-polyisoprene-block-polystyrene is another example of a styrene block copolymer:

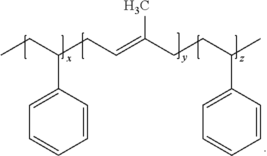

In one example, the hardness of this block copolymer ranges from about 25A to about 70A. In one specific example, the polystyrene-block-polyisoprene-block-polystyrene has the following properties: density ranging from about 0.80 g/mL to about 0.98 g/mL at 25° C.; and from about 5 wt % to about 30 wt % styrene content and from about 60 wt % to about 90 wt % isoprene content. It is to be understood that other percentages of the monomers may be used, and that a higher isoprene content results in a softer copolymer or block copolymer.

In any of these examples, the styrene polymer, styrene copolymer, or styrene block copolymer may be incorporated into a stock solution at 2.5-40 wt % so that a viscosity of the solution ranges from about 20 cP to about 20000 cP (measured at 25° C.).

The polymer matrix 16 should also take up (absorb) little to no water, as water and humidity may react with the NO donor particles 18 and prematurely release NO and degrade the NO donor particles 18. In an example, the water uptake exhibited by the polymer matrix 16 ranges from about 0.0 mg H₂O/mg polymer to about 0.5 mg H₂O/mg polymer. In one specific example, the water uptake exhibited by the polymer matrix 16 is about 0.2±0.18 mg H₂O/mg polymer.

Additionally, the polymer matrix 16 should not absorb the majority of the activating wavelengths for the solid, light sensitive NO donor particles 18. In one example, the polymer matrix 16 has >60% transmission of light with wavelengths in the range of 250 nm to 600 nm, to allow the NO donor particles 18 to be photolyzed to release the desired NO product.

The solid, light sensitive NO donor particles 18 are both in solid form and are light sensitive. By “solid form,” it is meant that the NO donor particles 18 are not a liquid or a fluid, but rather, are firm and stable in shape. In some examples, the NO donor particles 18 are in crystalline or powder form. By “light sensitive,” it is meant that the NO donor particles 18 are photolyzable, i.e., are capable of undergoing photolysis when exposed to an activating wavelength or wavelengths of light. In particular, the NO donor particles 18 are capable of releasing NO gas molecules when exposed to the activating wavelength or wavelengths of light. Examples of the solid, light sensitive NO donor particles 18 include light sensitive S-nitrosothiols. Some specific examples of light sensitive S-nitrosothiols are selected from the group consisting of S-nitroso-N-acetyl-penicillamine (SNAP), S-nitrosoglutathione (GSNO), S-nitroso-N-acetylcysteine (SNAC), S-nitroso-3-β-mercaptopropionic acid (SN3-MPA), and combinations thereof. These light sensitive S-nitrosothiols are capable of undergoing photolysis when exposed to activating wavelength(s) ranging from 250 nm to 600 nm. As specific examples, light emitting diodes (LEDs) emitting anywhere from 300 nm to 565 nm wavelengths may be used to activate the SNAP and GSNO crystals. As other examples, a bulb or laser emitting anywhere from 250 nm to 600 nm wavelengths may be used.

The light sensitive NO donor particles 18 have a volume-weighted mean diameter of 50 μm or less. In some examples, the volume-weighted mean diameter is less than 50 μm. In an example, the volume-weighted mean diameter ranges from about 0.1 μm to 50 μm. In another example, the volume-weighted mean diameter ranges from about 5 μm to less than 40 μm. In another example, the volume-weighted mean diameter ranges from about 1 μm to less than 25 μm. In still another example, the volume-weighted mean diameter ranges from about 0.5 μm to less than 5 μm.

In one example, a larger form (e.g., crystals) of the NO donor material (which forms the particles 18) are exposed to grinding and/or milling processes, which may be manual or automated to obtain smaller particles. In one example of automated milling, the NO donor material may be processed by ball mill. The milling processing can range from about 1 hour to about 48 hours to achieve a desired mean particle size distribution. The milling media can be composed of stainless steel, zirconia, ceramic, or other inert material. The milling container can be composed of stainless steel, zirconia, ceramic, HDPE, PTFE, or other inert material. The NO donor material can be milled as a dry powder formulation or milled as a slurry, which contains the NO donor material and a solvent or the NO donor material, a solvent, the polymer matrix 16, and one or more of the additives set forth herein. In yet another example of automated milling, the NO donor material may be processed by jet-mill. In one example the powder feed rate to the mill may range from 1 g to 6 kg per hour with feed and grind pressures ranging from 10 PSI to 20 PSI to achieve the desired mean particle size distribution. While examples have been provided, it is to be understood that the process parameters may be altered depending upon the type of equipment used.

After grinding and/or milling, the small particles may be sifted through a sieve screen in a humidity-controlled environment (e.g., less than 10% relative humidity) in order to avoid particle agglomeration. The size of the pores of the sieve screen may be 50 μm or smaller, so that each of the particles in the sieved sample has a particle size less than the pore size. In some examples, the ground particles may be sieved twice, where the first sieve screen is used to filter out particles that are too large and the second sieve screen is used to filter out particles that are too small. In one example, the first sieve screen may have a pore size of 25 μm and the particles that pass through the pores are collected. The collected particles have a diameter less than 25 μm. The collected particles are then filtered using a second sieve screen having a pore size of 10 μm. The particles that do not pass through the second sieve screen are collected and used as the solid, light sensitive NO donor particles 18. In this particular example then, the solid, light sensitive NO donor particles 18 have a diameter ranging from 10 μm to 25 μm. In other examples, the solid, light sensitive NO donor particles 18 have a diameter ranging from about 0.5 μm to 25 μm. In still other examples, the solid, light sensitive NO donor particles 18 have a diameter ranging from about 0.1 μm to about 5.0 μm.

The solid, light sensitive NO donor particles 18 are distributed throughout the polymer matrix 16. The method described herein enables the relatively uniform distribution of the solid, light sensitive NO donor particles 18 throughout the polymer matrix 16.

The weight ratio of the solid, light sensitive NO donor particles 18 to the polymer matrix 16 ranges from about 0.1:1 to about 50:1. In one example, the weight ratio of the solid, light sensitive NO donor particles 18 to the polymer matrix 16 ranges from about 1:1 to about 35:1. In still other examples, the weight ratio of the solid, light sensitive NO donor particles 18 to the polymer matrix 16 ranges from about 0.2:1 to about 9:1.

Some examples of the NO donor film 14 or the NO releasing film 10″ consist of the polymer matrix 16 and the NO donor particles 18. Other examples of the NO donor film 14 or the NO releasing film 10″ include one or more additives, such as NO₂ scrubber particles, radical stabilizers, dispersants/wetting agents, and/or anti-skinning agents. Example NO₂ scrubber particles include ascorbic acid, soda lime, calcium hydroxide, and/or sodium hydroxide. Example radical stabilizers include 2,6-Di-tert-butyl-methoxyphenol and/or 4-tert-butylcatechol. In an example, a mole ratio of 0.1:1 up to 1:1 of NO₂ scrubber particles or radical stabilizers to NO donor particles 18 may be present in the slurry and the NO donor film 14 or NO releasing film 10″. In other examples, an additional coating (not shown) containing the NO₂ scrubber particles or radical stabilizers may be positioned over the NO donor film 14 and beneath the protective coating 20 (if present). Example dispersants and pigment wetting agents include Kutsumoto Chemicals DISPARLON® series of dispersants or Lubrizol SOLSPERSE™ series of dispersants. In an example, a mass ratio of 1% to 25% of dispersant, with respect to NO donor particle mass, may be present in the coating slurry and NO donor film 14 or NO releasing film 10″. As another example, the mass ratio of 1% to 12.5% of dispersant, with respect to NO donor particle mass, may be present. The stock solution of the dispersant may be a diluted form (e.g., 50% active dispersant in n-butylacetate), and thus the amount may be adjusted in accordance with the concentration in the stock solution. In another example, anti-skinning agents, (e.g. ASCININ® from Milliken) may be present in mass ratios of 0.05% to 2.0% with respect to total slurry mass in the coating slurry and with respect to a total film mass in the NO donor film 14 or the NO releasing film 10″. In still another example, the slurry or film 14 or 10″ includes from about 0.2% to about 0.6% based on a total mass of the respective slurry or film 14 or 10″.

While a single layer of the NO donor film 14 is shown in FIG. 1A, it is to be understood that any number of layers may be applied to form the film 14. Thus, in some examples, the film 14 may include multiple layers stacked on top of each other. The NO donor film 14 may also be positioned on one side of the substrate 12, or on both sides of the substrate 12. When positioned on both sides of the substrate 12, each side may include a single layered or multi-layered NO donor film 14. In one specific example, the substrate 12 includes a multi-layered NO donor film 14 (e.g., including 2 layers) on each side.

The NO releasing film 10 shown in FIG. 1A may also include an NO permeable and light transparent film 20 positioned on the NO donor film 14. This example film 20 is permeable to nitric oxide. As such, NO that is released from the solid, light sensitive NO donor particles 18 can pass through nanopores or micropores of the NO permeable and light transparent film 20 into a recipient gas stream. This example film 20 is also transparent to the activating wavelength(s) of light used to release the nitric oxide from the solid, light sensitive NO donor particles 18. As such, in this example, light of desirable wavelength(s) may be transmitted to the solid, light sensitive NO donor particles 18 through the film 20. As examples, the film 20 may be transparent to one or more activating wavelengths of light ranging from about 250 nm to about 600 nm.

When included, the NO permeable and light transparent film 20 serves as a protective layer, helping to ensure that the NO donor particles 18 are not removed by abrasion or physical contact of the film 10 with other surfaces (the film itself, rollers, guides, or the like) during use.

An example of the NO permeable and light transparent film 20 includes the polymer matrix 16 without the inclusion of the solid, light sensitive NO donor particles 18. The NO permeable and light transparent film 20 may help immobilize the NO donor layer 14 to the substrate 12. The NO permeable and light transparent film 20 may also increase the NO releasing film's robustness, as it can reduce the propensity of NO donor layer 14 removal when the NO releasing film 10 is brushed or scraped.

While a single layer of the NO permeable and light transparent film 20 is shown in FIG. 1A, it is to be understood that any number of layers may be included to form the film 20. Thus, some examples of the film 20 include multiple layers stacked on top of each other.

The total thickness of the NO donor film 14 and the NO permeable and light transparent film 20 or the NO releasing film 10″ should be at most 2 mm. In FIG. 1A, the layer(s) of the films 14 and 20 are thin enough that the overall thickness of the films 14 and 20 is 2 mm or less. Thinner film(s) 14, 14 and 20, or 10″ enable the desired light penetration and can also be coiled/spooled without cracking. In one example, the thickness of the NO generating film 10 ranges from about 0.005 mm to about 1 mm. In another example, the thickness of the film 14, 14 and 20, or 10″ ranges from about 0.25 mm to about 1 mm.

The length of the NO releasing film 10, 10″ may range from about 10 m to about 50 m. When rolled, the diameter of the rolled NO releasing film 10, 10″ may range from about 8 cm to about 10 cm. These dimensions may be smaller, depending upon the size of the cartridge in which the film 10, 10″ is to be introduced.

### Method for Making the NO Releasing Film

An example method for making the NO releasing film 10 or 10″ includes dissolving the polymer matrix 16 into a solvent mixture, thereby producing a polymer solution; mixing the solid, light sensitive NO donor particles 18 into the polymer solution, thereby producing a coating slurry, wherein a volume-weighted mean diameter of the solid, light sensitive NO donor particles 18 is 50 μm or less; depositing the coating slurry on a support selected from the group consisting of a temporary substrate 12′ (see FIG. 1B) and the substrate 12 (which is impermeable to the solvent mixture); and evaporating the solvent mixture, thereby producing the NO donor film 14 on the support.

The polymer matrix 16 is dissolved into the solvent mixture to form a polymer solution. Dissolution of the polymer in the solvent enables rapid mixing, and homogenization of the dissolved polymer with other slurry components (if included). The solvents of the solvent mixture are selected so that they dissolve the polymer matrix 16 and so that they have no effect on the underlying support (e.g., substrate 12 or temporary substrate 12′). As such, the substrate 12 or 12′ is chemically inert to the solvent mixture. In other words, the solvent mixture does not dissolve the substrate 12 or 12′ or penetrate into the fibers of the substrate 12 or 12′. Examples of the solvent mixture may include 1:4 to 4:1 wt:wt of a low vapor pressure solvent (e.g., cyclohexanone) to a high vapor pressure solvent (e.g., ethanol, tetrahydrofuran) or 90:10 to 10:90 n-butylacetate:ethanol. Other examples of solvents that may be used neat or in the solvent mixture include methyl alcohol, isopropyl alcohol, ethyl acetate, hexanes, xylenes, toluene, decane, and dodecane.

The amount of the polymer matrix 16 in the solvent mixture may range from about 2.5 wt % to 40 wt %, based on the total weight of the resulting polymer solution. The weight percentage of polymer may vary depending upon the solubility of the particular polymer in the solvent mixture.

In a first example, the polymer solution may be used as is to create a coating slurry for producing the film 14 or 10″, or, in a second example, the polymer solution may be prepared as a stock solution for preparation of a coating slurry that contains a lower wt % of the polymer than the stock solution. In the first example, the amount of the polymer matrix 16 in the solvent mixture ranges from about 2.5 wt % to about 15 wt % (based on the total weight of the polymer solution) and is used undiluted. In this example, the solid, light sensitive NO donor particles 18 are added to the undiluted polymer solution to produce the coating slurry. In the second example, the initial polymer solution is prepared as a stock solution containing from about 10 wt % to about 40 wt % of the polymer matrix 16. Some of the stock solution is combined with additional solvent and the solid, light sensitive NO donor particles 18 (described below) to produce a final coating slurry where the polymer content ranges from 2.5 wt % to about 25.0 wt % based on the total weight of the coating slurry.

Once the polymer matrix 16 and the solvent mixture are combined, they may be stirred, shaken, or otherwise mixed until the polymer matrix 16 is completely dissolved. This forms the polymer solution. While several examples have been provided, it is to be understood that the weight percentage of the polymer matrix 16 in the polymer solution may also depend upon the deposition technique that is to be used to apply the slurry. For example, when a pneumatic spraying application is to be used, the concentration of the polymer matrix 16 in the polymer solution may be at the middle to lower end of the given range so that the solution can be sprayed. For another example, when a knife-edge film application is to be used, the concentration of the polymer matrix 16 in the polymer solution may be at the middle to upper end of the given range so that the viscosity of the polymer solution and the final coating slurry is increased.

The solid, light sensitive NO donor particles 18 may then be added to the polymer solution to form the coating slurry. The concentration of the solid, light sensitive NO donor particles 18 in the coating slurry ranges from about 0.5 wt % to about 70 wt %, based on the total weight of the coating slurry. It is to be understood that the amount of the NO donor particles 18 in the coating slurry may also depend upon the deposition technique that is to be used to apply the coating slurry. In one example, the amount of the solid, light sensitive NO donor particles 18 in the coating slurry ranges from about 0.5 wt % to 20.0 wt % (based on the total slurry weight), while in another example, the concentration of the NO donor particles 18 in the slurry ranges from about 15.0 wt % to 60.0 wt % (based on the total slurry weight).

If included, the additive(s) may be added to the polymer solution before, with, or after the NO donor particles 18. As such, one example of the method further includes adding an additive to the polymer solution, wherein the additive is selected from the group consisting of NO₂ scrubber particles, a radical stabilizer, a dispersant, an anti-skinning additive, and combinations thereof. In one example, the dispersant is added to the solvent mixture, and then the polymer matrix 16 is added.

The slurry may be sonicated or blended in a homogenizer to help ensure that the NO donor particles 18 and any additives are substantially uniformly dispersed.

In some instances, the coating slurry consists of the solvent, the polymer 16, and the NO donor particles 18. In other instances, the slurry consists of the solvent, the polymer 16, the NO donor particles 18, and the additive(s).

The viscosity of the slurry may range from about 1 mPa-s to about 10000 mPa-s at room temperature (measured with a rotary viscometer).

When the support is the substrate 12, the slurry may be applied to the substrate 12 using any suitable deposition technique. With any of the techniques and when the substrate 12 is porous, it is believed that at least some of the slurry is able to penetrate into the nanopores or micropores of the substrate 12 and to form a relatively homogeneous layer of the slurry over the entire surface of the substrate 12.

In one example, a pneumatic paint spray nozzle is used to spray the slurry over the substrate 12. In another example, a knife-edge film coating technique is used to apply the slurry. With the knife-edge film coating technique, the substrate 12 can be dipped in a reservoir of the slurry, or the slurry can be poured or pipetted onto the surface of the substrate 12. A knife edge or doctor blade made of glass or solvent-resistant plastic is then passed over the slurry with the application of light pressure. By “light pressure,” it is meant that the pressure that is applied is not enough to completely scrape the slurry from the surface but is enough to remove excess slurry from the surface. As an example, the light pressure applied by the knife edge or doctor blade ranges from greater than 0 bar to about 5 bar. In still another example, the slurry can be applied via the flow channel of a slot die coater so that a reproducible layer is deposited on the substrate 12. In another example, the slurry can be applied via a gravure rod with a doctor blade such that a reproducible layer is deposited on the substrate 12.

The solvent mixture is then evaporated from the applied slurry layer. Evaporation may take place at ambient temperatures (from about 22° C. to about 26° C.), or may be accelerated by brief exposure of the applied slurry layer to heat up to 120° C. As examples, a suitable drying temperature for a slurry containing: ethanol, toluene, or n-butyl acetate ranges from 30° C. to 120° C. with drying times ranging from 10 seconds to 360 seconds, or ranges from 22° C. to 26° C. with drying times ranging from 12 hours to 24 hours.

In some examples, evaporation is accelerated at a temperature ranging from 40° C. up to 100° C. In one example, quick flash drying at temperatures ranging from 70° C. up to 120° C. may be used to accelerate evaporation of the solvent mixture. Prolonged exposure to higher temperatures could cause the NO donor particles 18 to decompose. In an example, brief exposure ranges from about 30 s to about 360 s.

It has been observed with some polymer matrices 16, that both drying conditions and polymer may cause a dense layer of the polymer matrix 16 at the film 14 surface. This dense layer acts like a crust that can trap NO gas released from the NO donor particles 18, that can retain solvent residuals in the film 14, and that can promote the formation of excess NO₂ gas byproduct. The weight percent of the polymer matrix 16 in the coating slurry, vapor pressure of the solvent, drying time, and drying temperatures can all affect the polymer's propensity to form this dense crust layer. Specifically, when thick films are rapidly dried at elevated temperatures, the surface of the film may dry first, causing skin formation prior to complete solvent evaporation from the remainder of the film. When the content of the polymer matrix 16 in the polymer solution and slurry is greater than or equal to (≥) 20 wt % (with respect to the weight NO donor particles in the slurry), or when the total film thickness is greater than or equal to 20 μm, a stepwise ramping drying temperature or an extended drying time may be utilized to prevent the formation of the dense crust layer.

As the solvent mixture evaporates, the polymer matrix 16 remains along with any other additives, if included, providing a dried polymeric matrix for the solid, light sensitive NO donor particles 18. The polymer matrix 16 securely anchors to the substrate 12 or base binding layer 13, while also providing the NO donor film 14 with flexibility so that it can bend and move with the substrate 12 without cracking or breaking.

With the porous substrates 12, the NO donor film 14 may be present in at least some of the pores located at/near the substrate surface upon which the slurry was deposited, and across the surface.

The process for generating the NO donor film 14 may be repeated any number of times to generate several layers stacked on top of one another, thus creating a multi-layered NO donor film 14. In this example, at least partial evaporation of one layer takes place before the application of more slurry. At least partial evaporation reduces the amount of liquid or eliminates the liquid, the presence of which can result in uneven film coating.

Some examples of the method further include forming a base binding layer 13 on the substrate 12 before forming the NO donor film 14. To form the base binding layer 13, a polymer solution without any of the solid, light sensitive NO donor particles 18 therein is deposited over substrate 12 before the slurry is applied, and the solvent mixture is evaporated. The polymer solution may be applied using the same techniques for applying the slurry, and evaporation may be allowed to occur or accelerated as described for the slurry. The polymer solution may include the same polymer that is used for the polymer matrix 16 or any other polymer disclosed herein for the base binding layer 13. The polymer solution used to form the base binding layer 13 may also include any of the additives set forth herein.

After the single layer or multi-layered NO donor film 14 is generated, the method may further involve forming the NO permeable and light transparent film 20 over the layer(s) making up the NO donor film 14.

To form the NO permeable and light transparent film 20, the polymer solution without any of the solid, light sensitive NO donor particles 18 therein is deposited over the NO donor film 14, the solvent mixture is evaporated. The polymer solution may be applied using the same techniques for applying the slurry, and evaporation may be allowed to occur or accelerated as described for the slurry. The polymer solution used to form the NO permeable and light transparent film 20 may also include any of the additives set forth herein.

The process for generating the NO permeable and light transparent film 20 may be repeated any number of times to generate several layers stacked on top of one another, thus creating a multi-layered NO permeable and light transparent layer 20. In this example, at least partial evaporation of one layer takes place before the application of more polymer solution. At least partial evaporation reduces the amount of liquid or eliminates the liquid, the presence of which can result in uneven film coating.

Another example method uses the temporary substrate 12′ instead of the substrate 12. This example method includes dissolving the polymer matrix 16 into a solvent mixture, thereby producing a polymer solution; mixing solid, light sensitive NO donor particles 18 into the polymer solution, thereby producing a coating slurry; casting the coating slurry on a temporary substrate 12′; and evaporating the solvent mixture, thereby generating an NO donor film 10′ that is removable from the temporary substrate 12′. In this example method, the polymer matrix 16, the NO donor particles 18, and polymer solution may be any of the examples set forth herein.

This method generates an NO releasing film 10′ (similar to film 14) that can be removed from the temporary substrate 12′. The temporary substrate 12′ may be any material from which the film 10′ can be removed. In one example, the NO releasing film 10′ can be peeled off of the temporary substrate 12′. In an example, the temporary substrate 12′ is stainless steel. Other suitable temporary substrate materials include TEFLON® (polytetrafluoroethylene from DuPont) or TEFLON® coated material or ceramics. With the temporary substrate 12′, the adhesion described between the polymer matrix 16 and the substrate 12 does not take place.

In this example method, the slurry preparation, deposition, and evaporation may be performed as described herein. In one specific example, the slurry is cast on the temporary support 12′.

Once formed, the NO releasing film 10′ can be removed from the temporary substrate 12′. Removal may be accomplished, for example, by peeling the NO releasing film 10′ from the temporary substrate 12′.

### Nitric Oxide Releasing System and Gas Delivery Device

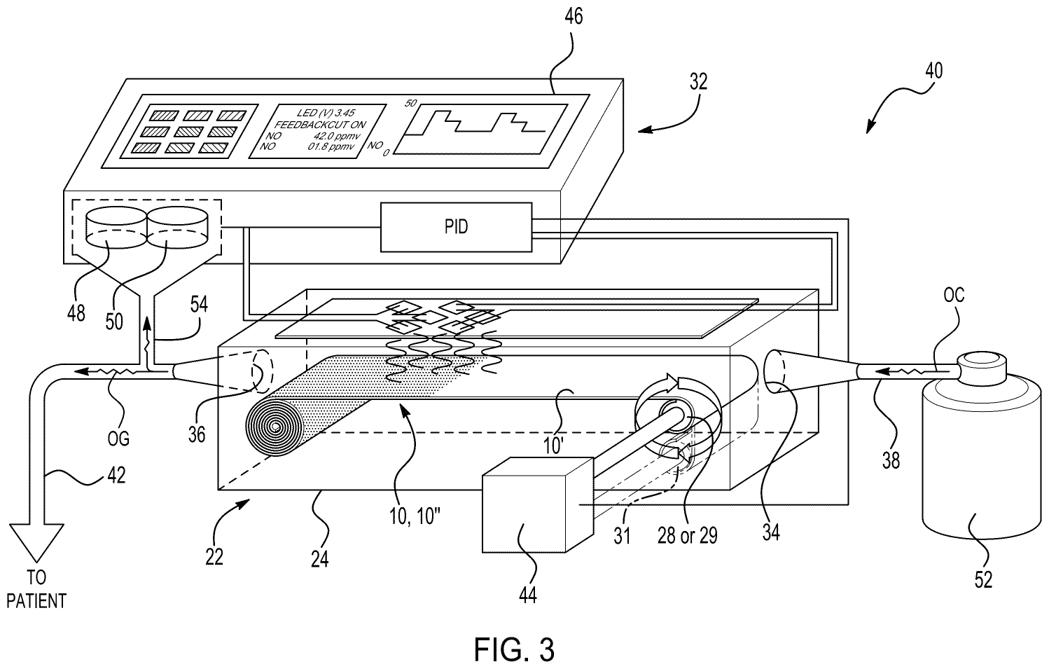

The nitric oxide (NO) releasing film 10, 10″ may be part of a nitric oxide releasing system. An example of the system 22 is shown in FIG. 2. The NO releasing system 22 includes a chamber 24; a spooling system including a supply reel 26 and i) a motor-controlled pick-up reel 28 or ii) a transfer reel 29 and a waste apparatus 31 (shown in phantom in FIG. 3); the NO releasing film 10, 10″ wound on the supply reel 26 and having an end attached to the motor-controlled pick-up reel 28 or to the waste apparatus 31; and a light source 30 operatively positioned to selectively expose a portion of the NO releasing film 10, 10″ to light (hv) to generate NO gas.

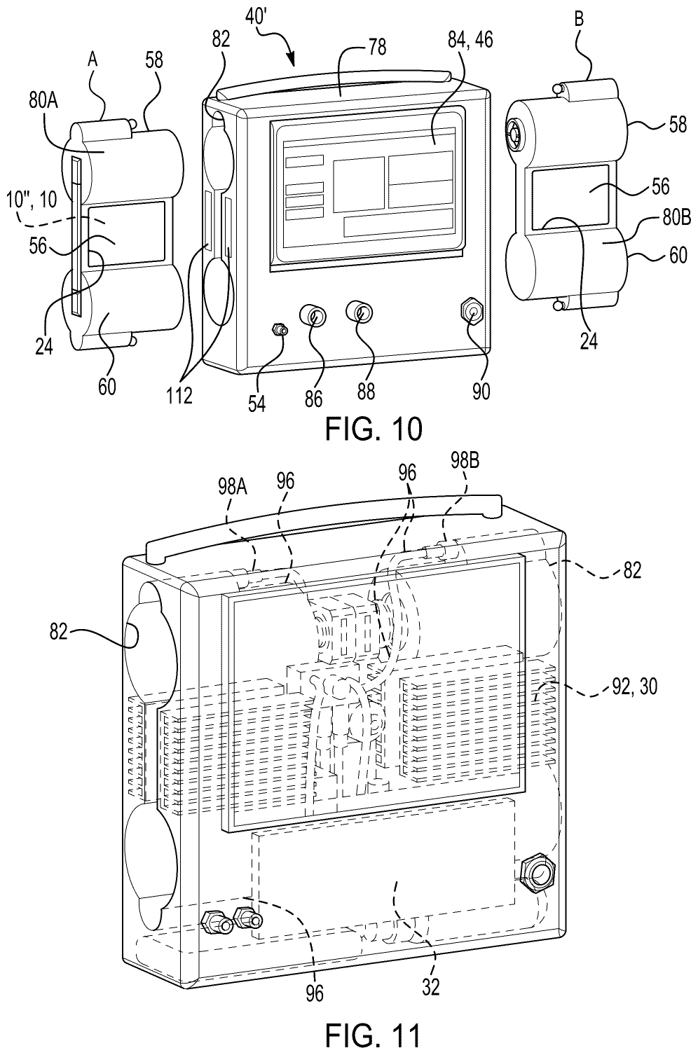

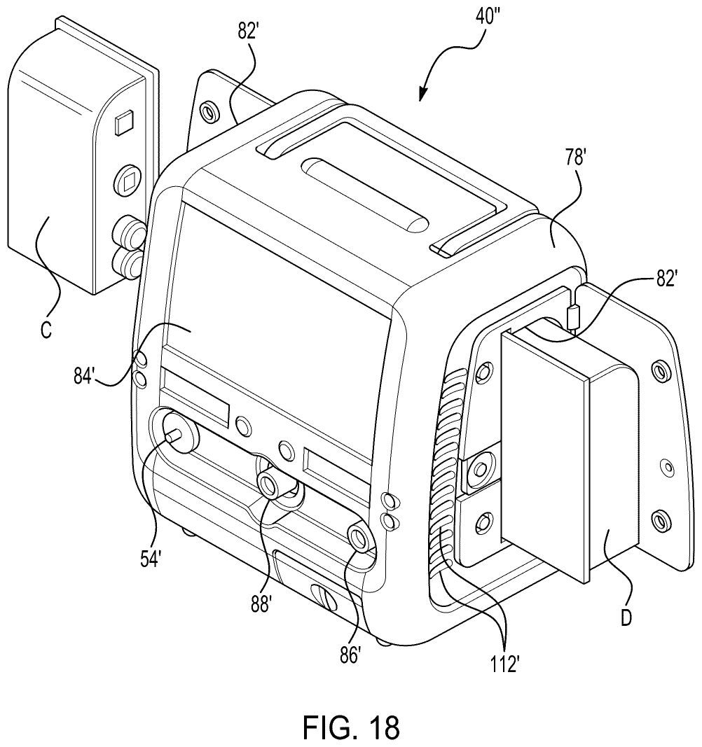

The NO releasing system 22 may be part of a gas delivery device. An example of the gas delivery device 40 is shown in FIG. 3. More specific examples of the gas delivery devices are shown in FIG. 10 and FIG. 18. As shown in FIG. 3, the gas delivery device 40 includes the NO releasing system 22, an inspiratory gas conduit 38 operatively connected to the chamber 24 to introduce an oxygen-containing gas (shown as “OC” in FIG. 3) and form an output gas (shown as “OG” in FIG. 3) including the NO gas; and an outlet conduit 42 to transport a stream of the output gas OG from the NO releasing system 22.

The system 22 and gas delivery device 40 will be described together in reference to both FIG. 2 and FIG. 3. While one example of the system 22 of FIG. 2 is shown in FIG. 3 (with the motor-controlled pick-up reel 28), it is to be understood that either of the example systems 22 described in reference to FIG. 2 could be used in the gas delivery device 40.

The NO releasing system 22 includes the chamber 24 where photolysis takes place (i.e., a photolysis chamber). The chamber 24 may be defined within a housing 80 that is made of any suitable material that is not permeable to oxygen-containing gas OC or to NO. If the light source 30 is positioned outside of the chamber 24 (as shown in FIG. 2), the housing 80 or a window 56 (see FIG. 10) defined in the housing 80 should be formed of a material that is transparent to the wavelength(s) of light hv emitted by the light source 30. In this example, the housing 80 that defines the chamber 24 may be formed of glass, poly(methyl methacrylate) (e.g., PLEXIGLAS® from Röhm), acrylonitrile butadiene styrene (ABS), low density polyethylene (LDPE), or UVT (Ultraviolet Transmitting) acrylic polymer, etc. If the light source 30 is positioned inside of the chamber 24, the housing 80 that defines the chamber 24 may be formed of a material that is non-transparent to the wavelength(s) of light hv emitted by the light source 30. In this example, the housing 80 may be formed of polytetrafluoroethylene (PTFE), high density polyethylene (HDPE), stainless steel, etc.

The chamber 24 provides a sealed environment where the released NO can mix with a desired inspiratory gas. The housing 80 includes both an inlet 34 and an outlet 36. The housing 80, and thus the chamber 24, may be sealed around the inlet 34 (connected to a conduit that is used to introduce the oxygen-containing gas OC) and the outlet 36 (connected to a conduit that is used to transport a stream of the output gas OG). The housing 80, and thus the chamber 24, may also be disposable so that the entire NO releasing system 22 can be discarded at the end of its useful life, or the housing 80, and thus the chamber 24, can include an opening through which the NO releasing film 10, 10″ can be replaced.

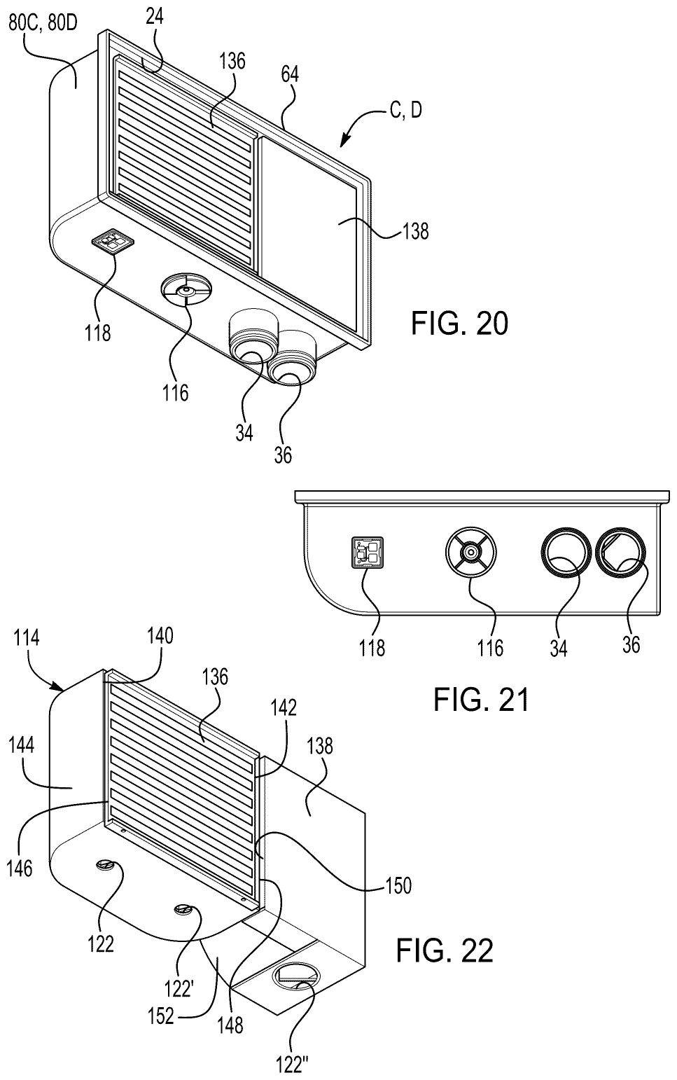

One example of the system 22 includes the supply reel 26 and the motor-controlled pick-up reel 28. In this example, both the supply reel 26 and the motor-controlled pick-up reel 28 are positioned within the chamber 24. The rods of each of the reels 26, 28 may be connected to a mechanism (not shown) that enables their operation. In one example, the supply reel 26 may include a stationary central rod that is securely attached to the housing 80 and an outer spool that is positioned on the stationary central rod and rotates about the stationary central rod. The fresh NO releasing film 10, 10″ is wound around the outer spool. The motor-controlled pick-up reel 28 includes a rotating rod that is operatively connected to a stepper motor 44 via a shaft or by other connection means such as a belt drive(s), gears and/or gear chain(s) (FIG. 3), which may be positioned inside the chamber 24 or outside of the chamber 24. When the stepper motor 44 is positioned outside of the chamber 24, the housing 80 may include a sealed opening for the drive/step motor coupler (see reference numeral 116 in FIG. 20) to extend therethrough and couple to the pick-up reel 28. The motor-controlled pick-up reel 28 collects the expended film 10′ (i.e., the film 10 or 10″ that releases NO as a result of light exposure).

The linear film advancement speed depends, at least in part, on the diameter of the axle of the motor-controlled pick-up reel 28. The following table illustrates example speeds for a 7.6 mm diameter axle.

TABLE 1

| | Linear Film Speed |
|---|---|
| Speed | (mm/step) |
| 1 | 0.119 |
| 2 | 0.239 |
| 4 | 0.477 |
| 8 | 0.955 |
| 10 | 1.193 |

In another example of the system 22, the supply reel 26 is used with a transfer reel 29 and a waste apparatus 31, which may be a waste reel or a waste container. Each of these components is also positioned within the chamber 24, and thus within the housing 80. In this example, the supply reel 26 is as described herein. The transfer reel 29 may also include a rotating rod that is operatively connected to a shaft of a stepper motor 44, which may be positioned inside the chamber 24 or outside of the chamber 24. Unlike the pick-up reel 28, the transfer reel 29 does not collect the expended film 10′. Rather, the transfer reel 29 may be configured to guide the expended film 10′ into the waste apparatus 31. As such, the transfer reel 29 is not secured to an end of the film 10, 10″, but rather allows the expended film 10′ to slide across its surface and into a suitably positioned waste container. The waste apparatus 31. As shown in FIG. 3, the waste apparatus 31 may be a roll that winds the expended film 10′. The waste apparatus 31 may alternatively be a container in which the expended film 10′ fold back and forth. Either example of the waste apparatus 31 may be removable from the system housing 80 so that the expended film 10′ can be removed at the end of its useful life.

The NO releasing system 22 also includes the NO releasing film 10 or 10″. The NO generating film 10, 10″ may be any of the examples described in reference to FIG. 1A or FIG. 1B. In the example shown in FIG. 2, the fresh NO releasing film 10 (whose NO donor film 14 is unexposed to light) or 10″ is wound on the supply reel 26 and has one end attached to the motor-controlled pick-up reel 28 so that when the motor-controlled pick-up reel 28 is operated, the NO releasing film 10 or 10″ moves in the direction of the motor-controlled pick-up reel 28. When the NO releasing film 10 is used, it is also positioned on the reels 26, 28 so that the NO donor film 14 faces the light source 30. The movement of the reels 26, 28 introduces fresh NO donor film 14 or NO releasing film 10″ in a position to be exposed to the light hv from the light source 30, which initiates NO release.

The NO releasing system 22 also includes the light source 30. Any light source may be used that is capable of emitting light that initiates photolysis of the solid, light sensitive NO donor particles 18. In other words, any light source 30 may be used that is capable of emitting the particular wavelength or wavelengths of light that cause the NO to be released from the solid, light sensitive NO donor particles 18. As such, the light source 30 may depend, in part, upon the NO donor particles 18 used and the desired rate of NO release. As examples, the light source 30 may be a high intensity light emitting diode (LED), a laser diode, a lamp, a bulb, etc. Suitable LEDs may be those having a nominal wavelength ranging or peaking at/from about 300 nm to about 700 nm, such as 340 nm, or 365 nm, or 375 nm, or 385 nm, or 395 nm, or 405 nm, or 450 nm, or 470 nm, or 527 nm, or 565 nm, or 595 nm. Suitable light source(s) could also be wide range LEDs that emit a board range of wavelengths, such as from 300 nm to 700 nm, simultaneously.

When the light source(s) 30 emits UV wavelengths (e.g., 100 to 400 nm), the NO releasing system 22 may also be self-sterilizing. The ultraviolet light has an anti-microbial effect and thus may sterilize the system 22 as well as the NO gas that is generated.

One or more light sources 30 may be used to release NO from the NO releasing film 10 or 10″. The use of multiple light sources 30 may enable further control over the NO release. In one example, one, or two, or eight, or more banks of LEDs may be used. For example, if higher levels of NO are desirable, all of the light sources 30 facing the NO donor film 14 of the NO releasing film 10 or facing the NO releasing film 10″ may be activated to emit light toward the film 14 or 10″, and if lower levels of NO are desirable, less than all of the light sources 30 may be activated. Additionally, the intensity of the light source(s) 30 may be adjusted to increase or decrease NO release, and to achieve substantially uniform illumination when an array of light sources 30 is utilized.

The light source(s) 30 is/are positioned to selectively expose the NO donor film 14 or NO releasing film 10″ to light hv. The light source(s) 30 may be positioned outside of a light transparent housing 80 or may be positioned inside of a transparent or non-transparent housing 80. In some examples, the light source(s) 30 may be attached to the housing 80 (e.g., either inside or outside). In these examples, and when the housing 80 is disposable, the light source(s) 30 may be disposed with the housing 80. In these examples, and when the housing 80 is not disposable (but rather receives a disposable NO releasing film 10 or 10″), the light source 30 may be reused with several different NO releasing films 10 or 10″. The light source(s) 30 may also be removable from the inside or outside of the housing 80 so that it/they can be replaced at the end of its/their useful life. In some other examples, the light source(s) 30 may be attached to a main body 78 of a gas delivery device 40, such as the example devices 40′, 40″ shown in FIG. 10 and FIG. 18, which receive an example of the NO releasing system 22. In these examples, the light source(s) 30 may not be directly attached to the housing 80, but may be positioned within the main body 78 to direct the light hv to the NO donor film 14 or NO releasing film 10″ inside the chamber 24 when operated. In these examples, the light source(s) 30 may be removable from the main body 78 so that it/they can be replaced at the end of its useful life.

When the light source(s) 30 is/are attached to the inside of the chamber 24 or to an interior surface of the main body 78, any adhesive or other suitable securing mechanism may be used to attach the light source(s) 30 to an interior wall or structure of the housing 80 or the main body 78. This adhesive may not be light transparent because it is not positioned between the light source(s) 30 and the film 14 or 10″. When the light source(s) 30 is/are attached to the outside surface of the housing 80 that defines the chamber 24 (as shown in FIG. 2), any light transparent adhesive or other suitable securing mechanism may be used to attach the light source(s) 30 to an exterior wall or structure of the housing 80. In these instances, it is to be understood that this securing mechanism will not block the light from reaching the NO releasing film 10 or 10″. The light source(s) 30 may also be operatively positioned outside of, but not attached to, the housing 80 as long as the output light is directed toward the NO donor film 14 of the NO releasing film 10 or toward the NO releasing film 10″.

The proximity of the light source(s) 30 and the NO releasing film 10 or 10″ may also be controlled. In an example, a desirable distance between the light source(s) 30 and the NO releasing film 10 or 10″ may range from about 1 mm to about 80 mm. In one example, the distance is about 30 mm.

The angle of the light source(s) 30 with respect to the surface of the NO releasing film 10 or 10″ may also be adjusted.

While not shown in either FIG. 2 or FIG. 3, it is to be understood that the NO releasing system 22 may also include a heat sink that is configured to cool the light source(s) 30. Examples of suitable heat sinks include those available from Wakefield-Vette (e.g., the round star LED boards heat sink or the radial fin heat sink). In one example, the heatsink is mounted on top of the light source(s) 30. The light source(s) 30 may also be mounted using other controller boards, and may utilize other heat sink/heat dissipation modules of other heat dissipating materials, such as aluminum 20 mm×20 mm×10 mm radiator-style Heat Sinks; extruded heat sinks fabricated from aluminum or aluminum alloy; bonded heat sinks fabricated from aluminum, aluminum alloy, copper or a combination; skived heat sinks fabricated from copper; forged heat sinks; or specialty CNC machined heat sinks. Heat dissipation may also be controlled with zinc oxide or ceramic thermal paste, re-directed airflow, one or more fans directed at the boards, or a circulating liquid or water-cooled circuit. In an example, the temperature within the chamber 24 may be maintained at about 25° C. to about 45° C., depending upon the intensity of the light source(s) 30 and the flow rate through the chamber 24. In one example, a flow rate of 4 LPM has a lower temperature within the given range and a flow rate of 0.5 LPM has a higher temperature within the given range.

The NO releasing system 22 may be part of a gas delivery device 40, as shown in FIG. 3. The gas delivery device 40 also includes electronic circuitry 32, which may be operatively connected to the light source(s) 30, the stepper motor 44, and the heat sink. The electronic circuitry 32 attached to the light source(s) 30 may control when the source(s) 30 is/are turned ON and OFF, the duration of an ON cycle, the intensity, the power surface density, etc. The electronic circuitry 32 attached to the stepper motor 44 may control when the reel 28 is turned ON and OFF, the duration of an ON cycle, the speed of the reel 28, etc.

An example of the electronic circuitry 32 includes Raspberry Pi based electronic boards and components. Another example of the electronic circuitry 32 includes custom printed circuit boards specifically designed to control electronics, such as the light sources 30 and stepper motor(s) 44, as well as other electronic components in the system 40. The electronic circuitry 32 may include components to store (i.e., computer memory; e.g.; SD cards, EEPROM, RAM, flash memory, etc.) and run (e.g., microcontrollers (MCU), graphics processing units (GPU), graphic card(s), etc.) a graphical user interface (GUI) 46 (FIG. 3) that provides a graphical readout of the data and contains the user controls to operate the system 22. The electronic circuitry 32 may also contain any type of display components (e.g., LCD or LED monitors), and/or a touchscreen display(s) and/or physical buttons to control the device and observe how the device 40 is functioning (e.g., visually monitor NO levels in ppm released from the NO releasing film 10 or 10″).

The electronic circuitry 32 may also be part of a sensing and feedback system (i.e., a monitoring system, also referred to herein as a feedback and sensing system) that includes NO and NO₂ sensors 48, 50 for detecting, respectively, the NO level and the NO₂ level (if any) in the output gas OG. These sensors 48, 50 may be in gas flow communication with a sampling line 54, which is in gas flow communication with the outlet conduit 42, which is connected to the outlet 36, as shown in FIG. 3. The sampling line 54 enables a portion of the output gas OG to reach the sensors 48, 50. It may be desirable to monitor the NO level for feedback control. The NO₂ sensor may be used for a safety threshold and may also be used for feedback control. In response to the measured NO and/or NO₂ levels, a PID controller (PID in FIG. 3) continuously adjusts the intensity of the light source(s) 30 based on the sensor readings and a user-input set point. As such, feedback control helps to avoid introducing NO₂ (nitrogen dioxide), which can be generated by the reaction of O₂ with NO, to a recipient/patient. The PID controller may respond to a control algorithm that automatically adjusts the intensity of the light source(s) 30 in response to the feedback so that desired NO and/or NO₂ levels may be obtained or maintained. In one example, the PID controller will continuously adjust the intensity of all the light sources 30 simultaneously, and in another example, only a user-selected subset the light sources 30 will be adjusted by the PID controller or control algorithm.

While not shown, it is to be understood that the sensing and feedback system may also include an oxygen sensor, gas flow sensors and/or differential pressure sensors in gas flow communication with the sampling line 54 and/or with the outlet conduit 42. The oxygen sensor may be used to measure the O₂ concentration in the output gas OG and/or for measuring the dilution of the air/O₂ in the outlet conduit 42. The gas flow sensors and/or differential pressure sensors may be used for integration of the sensing and feedback system with a ventilator. In particular, these sensors may be used to adjust the output gas OG to patient/ventilator breathing rate.

One example NO releasing system 22 or gas release and delivery device 40 includes at least two sets of sensors (48, 50 and an oxygen sensor) to monitor NO, NO₂ and O₂; and the feedback and monitoring system to receive data from the at least two sets of sensors. In this example, the feedback and monitoring system can compare measurements from the two or more sets of sensors to determine when sensor calibration should be run or to analyze sensor failure.

Still further, the NO releasing system 22 may include a manual or electronic backup system. This system enables the continued delivery of the NO when the gas(es) supplied by the ventilator is/are manually delivered. When the ventilator is undergoing maintenance or experiences a malfunction, the NO releasing system 22 can enter static mode, where the dosage of the NO that is generated and delivered is based on the last measurement from the feedback system before static mode is entered. As such, the same amount of NO can be continuously delivered for some predetermined amount of time.

When in use, the light source(s) 30 may be turned ON for any time interval, for example, 8 hours per NO releasing film 10 or 10″, and thus may photolytically release NO during this time interval. Longer time intervals, and thus longer NO release lifetimes, may be possible, depending upon the size of the substrate 12 and/or the amount of NO donor particles 18 in the NO releasing film 10 or 10″. During this time interval, the stepper motor 44 controls the reel 28 so that it is turned ON continuously to bring fresh NO donor film 14 or NO releasing film 10″ within proximity of the light source(s) 30. When it is desirable to stop releasing NO, the light source(s) 30 is/are turned OFF so that light hv is no longer emitted on the NO releasing film 10 or 10″ and the stepper motor 44 is turned OFF so that fresh NO releasing film 10 or 10″ is not wasted.

In one example of the method, the supply reel 26 is 1.2 mm in diameter, the motor-controlled pick-up reel 28 is 3.77 mm in diameter, and the rotation ranges from about 0.5° per second to about 5° per second. In another example, the rotation of the reels 26, 28 may range from about 0.2° per second to about 14.5° per second.

In another example of the method, the pick-up reel 28 does not rotate, and the NO releasing film 10 or 10″ remains stationary.

In general, the operation of the NO releasing system 22 depends upon several factors, including the loading of the NO donor particles 18, the flow rate of gas to the patient, and the level of NO being delivered. As mentioned, the process is feedback controlled and the parameters can be adjusted to achieve the maximum delivery of NO from a specific area of film 10 or 10″.

The NO gas released from the NO donor particles 18 permeates through the NO donor film 14 (and the NO permeable and light transparent film 20 if used) or through the NO releasing film 10″ and into the chamber 24.

The gas delivery device 40 shown in FIG. 3 also includes the inspiratory gas conduit 38 operatively connected to the chamber 24 (e.g., at inlet 34 of the housing 80) to introduce the oxygen-containing gas OC to the chamber 24. The oxygen-containing gas OC may be at least substantially pure oxygen gas (O₂) or air, or a hypoxic gas that includes oxygen. In this example, the oxygen-containing gas OC may be delivered from any suitable gas source 52 (e.g., compressed gas cylinder (not shown), a gas pump that delivers ambient air, an oxygen concentrator, a medical ventilator, etc.), which can regulate the flow of the oxygen-containing gas OC, or can be coupled to a flow controller to regulate the flow of the oxygen-containing gas OC into the inlet 34. Any suitable gas flow rate may be used. As an example, the flow rate of the oxygen-containing gas OC may range from about 50 mL/min to about 5 L/min. In another example, the source 52 or flow controller may regulate the flow of the oxygen-containing gas OC so that the output gas stream OG contains from about 20% oxygen to about 99.99% oxygen. In an example, 100% air saturation may be used as the oxygen-containing gas OC, which corresponds to about 10 mg/L (ppm) of O₂ in the output gas stream OG.

The inspiratory gas conduit 38 may be a tube that has low or no permeability to at least the oxygen-containing gas OC and the nitric oxide. Examples of suitable tubing material include poly(vinyl chloride) (PVC), polyurethane (PU), polyethylene (PE), fluorinated polymers (e.g., polytetrafluoroethylene (PTFE), fluorinated ethylene propylene (FEP)), polycarbonates (PC), etc.

In the chamber 24, the oxygen-containing gas OC mixes with the photolytically released NO gas to form an output gas stream OG. A stream of the output gas OG may exit the NO releasing system 22 through the outlet 36 into the outlet conduit 42. The outlet conduit 42 may be a tube that has low or no permeability to at least the oxygen-containing gas OC and the nitric oxide in the output gas OG. The length of the outlet conduit 42 may also be relatively short in order to avoid nitrogen dioxide (NO₂) formation before the stream is delivered to a desirable destination (e.g., a recipient, such as a human patient). Since the oxygen-containing gas OC is introduced prior to delivery to the recipient, the impact on the NO concentration is minimal or nil due to the short contact time between the NO and the oxygen-containing gas OC.

In some examples, the output gas OG stream may be transported as a result of pressure from the gas source 52, which may include a regulator to control the flow rate. In other examples, the output gas OG stream may be transported as a result of pressure from a vacuum positioned downstream.

The outlet conduit 42 may be operatively connected to a delivery conduit (not shown). The delivery conduit may be operatively connected to an inhalation unit (not shown), which is capable of transporting the output gas stream OG to a recipient/patient. The delivery conduit may be any suitable polymeric or other tubing that is impermeable to the output gas stream OG. In an example, the delivery conduit may also have a one-way valve so that the output gas stream OG does not flow back into the NO releasing system 22. The inhalation unit may be a face mask, a nasal cannula, or some other suitable apparatus for delivering the output gas stream OG to the airways of the recipient.

While not shown, it is to be understood that the gas delivery device 40 may also include a nitrogen dioxide (NO₂) absorption filter or scrubbing module. The NO₂ absorption filter may be positioned in the delivery conduit to receive the output gas stream OG before it is delivered to the inhalation unit, and ultimately, to the recipient. Some examples of the NO₂ absorption filter remove at least some of the NO₂ from the output gas stream OG. As examples, a silica gel filter (with pre-conditioned silica particles) or a soda lime scrubber may be used as the NO₂ absorption filter. These filters may reduce the NO₂ to a level that is not physiologically relevant. Other examples of the NO₂ absorption filter convert the nitrogen dioxide back into nitric oxide. This conversion is desirable because no NO payload is lost in the form of scavenged (absorbed) NO₂, but rather is reduced back into NO. An example of this type of NO₂ absorption filter includes ascorbic acid (pure solid) or ascorbic acid impregnated silica particles.

Also while not shown, it is to be understood that the gas delivery device 40 may also include a dry scrubber to control the humidity (e.g., 5% to 90%) within the chamber 24. Examples of suitable scrubber materials include soda lime (most effective at higher humidity), calcium hydroxide (Ca(OH)₂) (effective at high humidity), sodium hydroxide (NaOH) (effective at lower humidity), potassium hydroxide (KOH) (effective at lower humidity), calcium chloride (Ca(Cl)₂), or any combination thereof.

Other examples of the gas delivery device 40′, 40″ are respectively shown in FIG. 10 and FIG. 18 (although the conduits 38, 42 are not shown). In each example, the gas delivery device 40′, 40″ is a portable nitric oxide releasing device that includes a main body 78, 78′ that can receive two cartridges A, B, or C, D, each of which includes its own NO releasing film 10 or 10″. The cartridges A, B, C, D are replaceable, so that when spent, they can be removed and replaced. The cartridges A, B, C, D are examples of the NO releasing system 22.

Each cartridge A, B includes a housing 80A, 80B that defines regions 58, 60 for the supply reel 26 and pick-up reel 28 and a chamber (similar to chamber 24) where photolysis takes place. When inserted into the portable main body 78 and in operation, the regions 58, 60 are not exposed to the excitation light. A cover 64 (which, in some instances, is opaque (in cartridges A, B), and in other instances, is transparent (in cartridges C, D)) may be integrally formed with the housing 80A, 80B, or may be a separate piece that is secured to the housing 80A, 80B. In either instance, the cover 64 seals the regions 58, 60. As mentioned, in the cartridges A, B, the cover 64 prevents light from reaching the film 10, 10″ or 10′. Example of opaque cover materials include polytetrafluoroethylene (PTFE), high density polyethylene (HDPE), acrylic, polycarbonate (PC), stainless steel, etc. The cover 64 in the cartridges A, B may also include one or more light transparent or translucent windows 56 that is/are positioned adjacent to the chamber 24 so that light can be delivered to the NO releasing film 10 or 10″ within the chamber 24. Alternatively, the housing 80A, 80B may include an opaque outer shell which defines openings for receiving respective covers 64, and that has a frame that supports the transparent/translucent window(s) 56. In this example, the cover 64 does not include the window 56, but rather two separate covers 64 are used to seal the respective regions 58, 60. One window 56 may be used to allow the film 10 or 10″ to be exposed on a single side. Two windows 56 positioned on opposed sides of the chamber 24 may allow the film 10 or 10″ to be exposed to the excitation light simultaneously on both sides of the film 10 that is positioned on a light transparent substrate 12, or of the film 10″, or to respective films 10 that are applied to opposed sides of a non-light transparent substrate 12. A transparent version of the cover 64 is described in reference to the cartridges C, D.

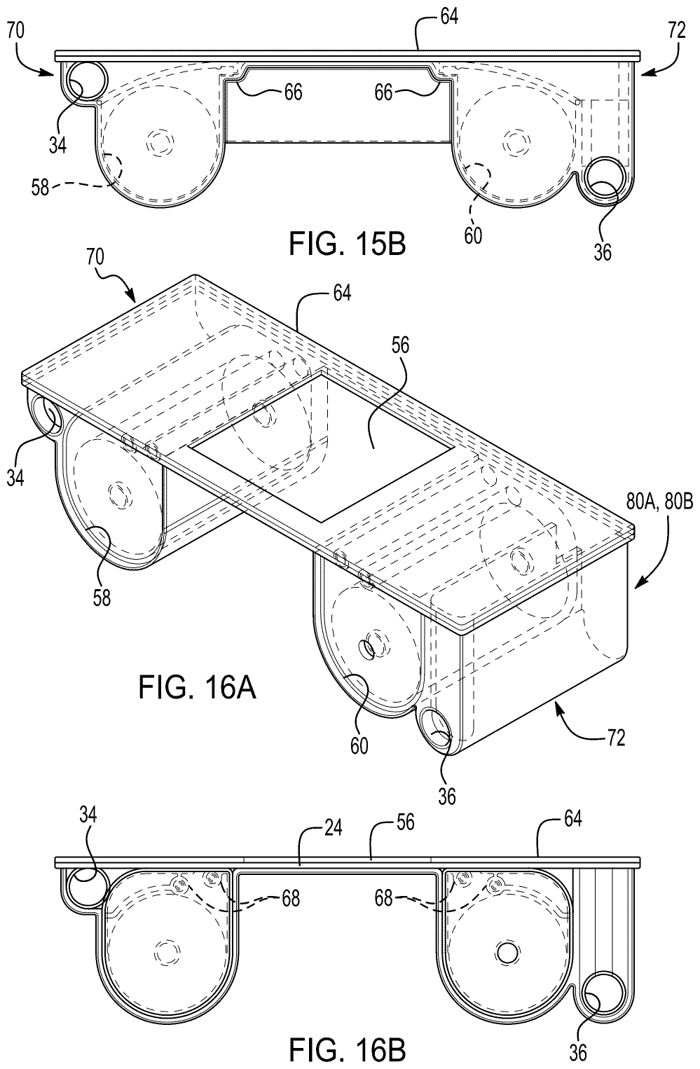

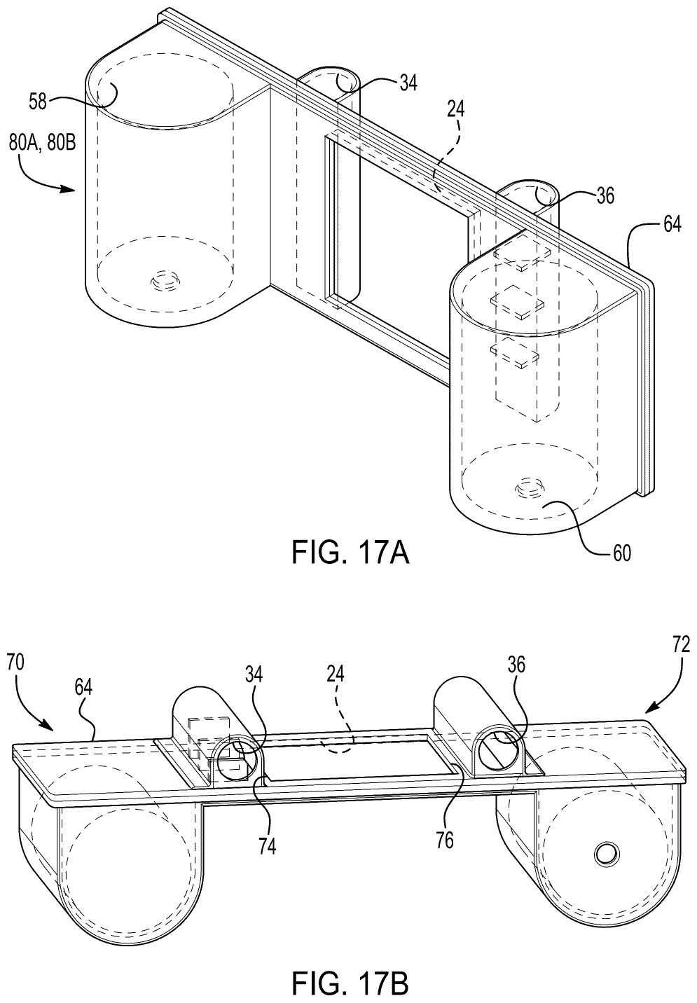

Perspective and side views of three examples of the cartridge housing 80A, 80B are shown, respectively, in FIG. 15A and FIG. 15B, and FIG. 16A and FIG. 16B, and FIG. 17A and FIG. 17B. To form the cartridge A, B, a cover 64 of the housing 80A, 80B is opened, the fresh NO releasing film 10 or 10″ is inserted into the operable position, and the cover 64 is closed, and in some instances sealed.

In some example housings 80A, 80B, the cover 64 can open so that a fresh NO releasing film 10 or 10″ can be loaded and a spent NO releasing film 10 or 10″ can be removed. In these examples, the housing 80A, 80B is reusable. It is to be understood, however, that the entire cartridge A, B (including the housing 80A, 80B) may be disposable. In these instances, the entire cartridge A, B can be removed and replaced.

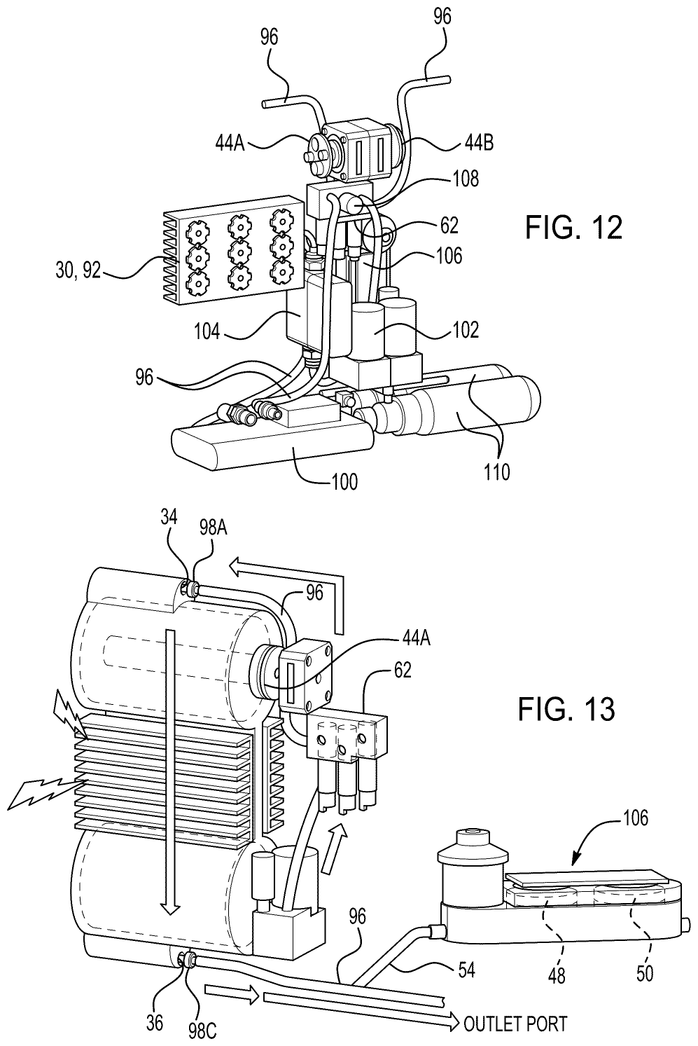

The interior chamber 24 that is adjacent to the window(s) 56 is where fresh film 10 or 10″ is introduced from the supply reel 26 and is exposed to light. This chamber 24 has dimensions that help to minimize NO₂ build up within the cartridge A, B (i.e., dead space within the chamber 24 is minimized while allowing the NO that is released from the releasing film to flow). For example, the thickness of the chamber 24 is less than 1 cm. The area leading to outlet(s) 36 of the housings 80A, 80B of the cartridges A, B, may also function as an absorber housing to absorb NO₂ from the NO containing gas stream that is exiting the cartridge A, B. For example, the section of the cartridge A, B leading to the outlet 36 may be formed of an NO₂ absorbing material or may have an NO₂ filter secured therein. Each of the cartridge housings 80A, 80B includes apertures where the stepper motor 44 (see FIG. 3, 44A and 44B FIG. 12, and 44A FIG. 13) can be operatively connected to the supply and/or pick up reels 26, 28.

In the example of the housing 80A, 80B of the cartridge A, B depicted in FIGS. 15A and 15B, a relief 66 is defined in the cartridge housing 80A, 80B between each of the regions 58, 60 and the chamber 24. The relief 66 allows the film 10 or 10″ to follow a smooth path into and out of the chamber 24 to make threading a leader on the film 10 or 10″ easier. In the example of the housing 80A, 80B of the cartridge A, B depicted in FIGS. 16A and 16B, film guides 68 are positioned to guide the film 10 or 10″ into or out of the regions 58, 60. In examples, the film guides 68 may include fixed shafts, ridges, ramps, posts, brushes, pads, idler rollers, or any mechanism for guiding the releasing film 10 or 10″. In the examples depicted in FIGS. 15A, 15B, 16A and 16B, the inlet 34 is defined on an inlet end 70 of the cartridge A, B, wherein the inlet end 70 is distal to the chamber 24. In the examples depicted in FIGS. 15A, 15B, 16A and 16B, the outlet 36 is defined on an outlet end 72 of the cartridge A, B, wherein the outlet end 72 is distal to the chamber 24. The inlet end 70 and outlet end 72 are opposed to each other. In the example depicted in FIGS. 17A and 17B, the inlet 34 is defined on the cover 64 adjacent to an inlet edge 74 of the window 56. In the example depicted in FIGS. 17A and 17B, the outlet 36 is defined on the cover 64 adjacent to an outlet edge 76 of the window 56.

The main body 78 of one example of the portable nitric oxide releasing device (gas delivery device 40′) is shown in FIG. 10 and the interior of the main body 78 is shown in FIG. 11. The main body 78 includes cartridge slots 82, a monitor 84 for displaying instructions and/or data to a user of the device 40′, a sampling gas port 54, an inlet port 86, and a gas outlet port 88. The device may also contain a power (PW) switch 90 to turn the device 40′ on or off. The switch 90 may selectively turn the monitor 84 on or off along with other selective electrical components (e.g., stepper motor 44A and 44B).

At the exterior of the device 40′, the gas inlet port 86 is capable of attaching to the inlet conduit 38 and to the gas source 52. Acceptable gas sources 52 are described above and include compressed oxygen/air sources, medical ventilators, etc. Within the device 40′, the gas inlet port 86 is fluidly connected to the inlets 34 of each of the cartridges A, B.

Also at the exterior of the device 40′, the outlet port 88 is capable of attaching to the outlet conduit 42. Within the device 40′, the outlet port 88 is fluidly connected to the outlet 36 of each of the cartridges A, B.

The gas generating device 40′ may also include one or more vents 112 positioned adjacent to the cartridge slots 82. In the example in FIG. 10, the vents 112 are positioned on either side of slot 82 and extend along the portion of the slot 82 where the window 56 of the cartridge A, B is introduced. The vents 112 enable air to flow in and out of the interior of the main body 78 in order to cool the light source(s) 30.

FIG. 11 depicts the interior of the cartridge slots 82, the positioning of the heat sinks 92 and light sources 30, the main printed circuit board (PCB) (i.e., the electronic circuitry 32), and the gas conduits 96. Some of the gas conduits 96 attach the inlet port 86 (FIG. 10) to respective connection ports 98A, 98B that lead to the respective cartridges A, B. Others of the gas conduits 96 attach a connection port 98C at the outlet 36 of the cartridge A, B to the outlet port 88 (see FIG. 13). When the cartridges A, B are inserted into the portable main body 78, the light sources 30 and heat sinks 92 are adjacent to the window 56 so that light can be directed to one or both sides of the film 10 or 10″ within the respective chambers 24 of the cartridge A, B.

FIG. 12 depicts some of the specific components that may be contained within the portable main body 78. These components include a battery 100, which may be operatively connected to the power switch 90. These components also include respective stepper motors 44A, 44B for independently operating the pick-up reels 28 of each of the cartridges A, B. These motors 44A, 44B are operated with power from the battery 100 and/or through external power supply source (e.g., AC to DC power supply, not shown).

FIG. 12 also depicts components of the sensing and feedback (or monitoring) system described herein. These components include the sampling pump 102, the sampling gas flow mechanism 104 (e.g., mass flow controller, or critical orifice), the sensor manifold 106, the solenoid manifold 62, and the flow change manifold 108.

The sampling pump 102 pumps a portion of the output gas OG through the sampling gas port 54. The sampling gas flow mechanism 104 may include a valve that opens the sampling gas port 54. Together, these components enable a sample of the output gas OG to be redirected to the sensor manifold 106, which holds the sensors 48, 50.

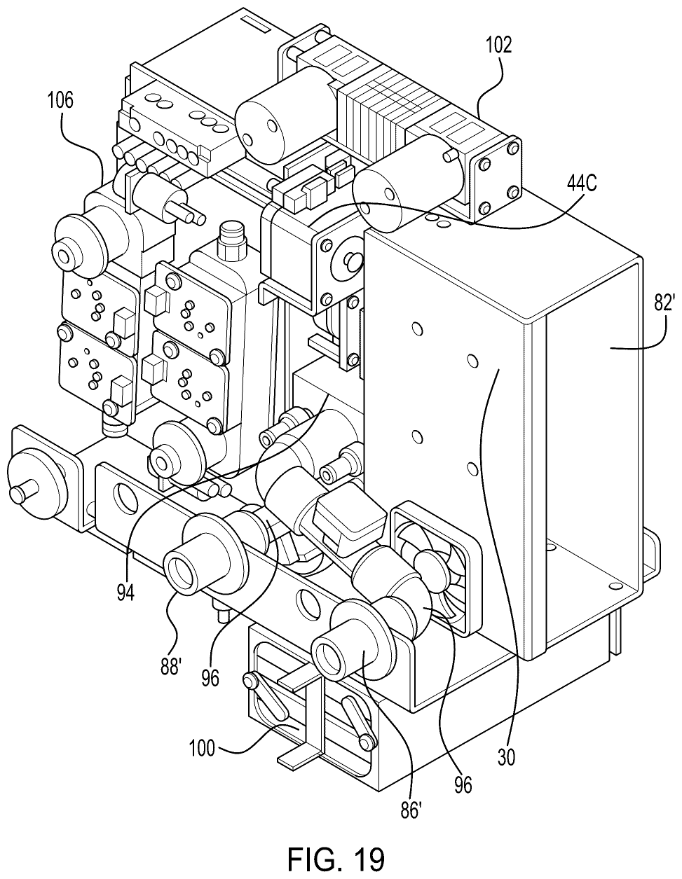

The flow change manifold 108 may instead be a selector value (see reference numeral 94 in FIG. 19) that allows for direction of gas flow to either cartridge A or B, or both cartridges A and B at the same time. In one example, the main body 78 includes two cartridge slots 82 that are to removably receive respective NO releasing systems (e.g., cartridges A and B, respectively); an oxygen-containing source 52 operatively connected to an inlet port 86 of the main body 78; and an automated switching valve (one example of 108) that, when in operation, directs flow from the oxygen-containing source 52 to one of the respective NO releasing systems, e.g., cartridge A or B to provide continuous NO delivery. For example, the automated switching valve can direct the flow from the source 52 to one or the other of the cartridges A, B as is required to continuously deliver NO. When switching flow from one cartridge A or B to the other B or A, the automated switching valve does not completely shut the flow off to the one cartridge A or b in order to maintain uninterrupted oxygen-containing gas flow to the device 40′. The automated switching valve can also isolate the cartridges A or B by completely shutting off the flow to one of the cartridge A or B while maintaining flow to the other cartridge B or A. Thus, the automated switching valve is configured to: provide some flow to one of the respective NO releasing systems (e.g., cartridge A) when switching flow to another of the respective NO releasing systems (e.g., cartridge B) to maintain uninterrupted flow through the device 40′; and shut off flow through either of the NO releasing systems (e.g., cartridge A or B) to isolate the one or the other of the respective NO releasing systems (e.g., cartridge A or B).

The flow change manifold 108 (or selector valve 94) interfaces with a gas flow sensor, which can measure the gas flow at the inlet and outlet of the cartridges A, B (or C and D described below) in order to detect a leak.

Also depicted are gas cylinders 110, which may be included for delivering gases during calibration. The calibration gas cylinders 110 may be housed with the device 40′, or stored externally (not shown) and connected when to be used for calibration. In the latter instance, the small calibration cylinders 110 of a known concentration of compressed NO or NO₂ gas will be temporarily connected to the device 40′ to calibrate the respective sensors (e.g., sensors 48, 50 in FIG. 2). As one example, the calibration gases include air, NO, or NO₂. Air will be taken from outside of device 40′ and introduced through the solenoid manifold 62 (which can toggle between the patient line and the calibration tanks 110) and air filter. NO or NO₂ calibration gas will be sent from desired cylinder 110 (see FIG. 12) to the sensor manifold 106. Thus, the solenoid manifold 62 is used for the calibration function. The solenoid manifold 62 re-directs the standard gas flow pathway from the NO releasing film 10 or 10″ to the sensors 48, 50 so that the calibration cylinder gas flow passes over the sensors 48, 50 instead. The cylinders 110 and solenoid manifold 62 may be part of an automated calibration system of the device 40′. The automated calibration system is operatively connected to the NO releasing system 22, and is operable via the electronic circuitry 32.

FIG. 13 depicts an example of the nitric oxide release and flow using one cartridge A when it is inserted into the device 40′, although some of the device components are not shown for clarity. To generate nitric oxide, the stepper motor 44A is initiated and controls the advancement of the film 10 or 10″ through the chamber 24. The light source(s) 30 are operated to expose the film 10 or 10″ in the chamber 24 to light (indicated by lightning bolt symbols in FIG. 13) through one or both windows 56. The oxygen-containing gas OC (e.g., from a ventilator) is introduced into the device 40′ through the gas inlet port 86, where it flows through gas conduits 96 and the connection port 98A to the inlet 34 where it will enter the cartridge A and mix with the released nitric oxide. The oxygen-containing gas OC flow route is controlled by the solenoid manifold 62 and the flow change manifold 108 so that, in this example, it enters the cartridge A. The combined gas stream, including the oxygen-containing gas OC and the generated NO gas, is output from the cartridge A through outlet 36. The combined gas stream is directed through additional gas conduits 96 to the outlet port 88. Some of the combined gas stream may be routed from the gas conduit 96 out through the sampling gas port 54 to the sensing and feedback system (including sensor manifold 106), so that the NO generation is controlled and is maintained at a desirable level.

Another example of the device 40″ is shown in FIG. 18. The main body 78′ is shown in FIG. 18 and the interior of the main body 78′ is shown in FIG. 19. The main body 78′ includes cartridge slots 82′, the monitor 84′ for displaying instructions and/or data to a user of the device 40″, the sampling gas port 54′, the outlet port 88′, and the gas inlet port 86′. The device 40″ may also contain a power (PW) switch to turn the device 40″ on or off and other switches to selectively turn the monitor 84′ on or off the monitor and/or to selectively turn other electrical components on or off.

At the exterior of the device 40″, the gas inlet port 86′ is capable of attaching to the inlet conduit 38 and to the gas source 52. Acceptable gas sources 52 are described above and include compressed oxygen/air sources, medical ventilators, etc.). Within the device 40″, the gas inlet port 86′ is fluidly connected to the inlets 34 of each of the cartridges C, D.

Also at the exterior of the device 40″, the outlet port 88′ is capable of attaching to the outlet conduit 42. Within the device 40″, the outlet port 88′ is fluidly connected to the outlet 36 of each of the cartridges C, D.

The main body 78′ of the gas generating device 40″ may also include one or more vents 112′ positioned adjacent to the cartridge slot 82′. In the example shown in FIG. 18, multiple vents 112′ are oriented parallel to the top and bottom surfaces of the main body 78′, and the line of vents 112′ extends along the length of one side of slot 82′. The vents 112′ enable air to flow in and out of the interior of the main body 78′ in order to cool the light source(s) 30.

FIG. 19 depicts the interior components of the device 40″.

In FIG. 19, the interior of a portion of one of the cartridge slots 82′ is depicted. While not shown, it is to be understood that at the back of the slot 82′, the inlet 34 and the outlet 36 of the cartridge C, D is able to operatively connect to respective connection ports that connect to gas conduits 96 and the inlet port 86′ and the outlet port 88′.

Adjacent to one side of the cartridge slot 82 is an LED board, which is one example of the light source 30 disclosed herein. The light source 30 is positioned along one side of the slot 82 so that the window 56 faces the light source 30 when the cartridge C, D is inserted into the slot 82′.

FIG. 19 also depicts the battery 100, the stepper motor 44C, the sensor manifold 106, a selector valve 94, and a sampling gas pump 102.

The battery 100 is similar to that shown and described in reference to FIG. 10.

The stepper motor 44C is operatively connected to the cartridge C when it is inserted into the slot 82′. Similar to the example shown in FIG. 12, it is to be understood that the device 40″ includes a second stepper motor (not shown) operatively connected to the cartridge D when it is inserted into its slot 82′. The stepper motors 44C in the device 40″ operate in the same manner as described herein.

The sensor manifold 106 is operable in the same manner as described for the device 40′.

The selector valve 94 may be operated as described herein in reference to FIG. 12. In short, the selector valve 94 is used to direct gas from source 52 (through conduit 38, input port 86′, inlet 34, and gas conduits 96) to either cartridge C (position 1) or cartridge D (position 2), or both cartridge C and cartridge D (position 3) at the same time. The selector valve 94 can also be used to close off the cartridges C, D from exposure to ambient air in a fourth position for storage.

The sampling gas pump 102 may be operated as described herein in reference to FIG. 12.

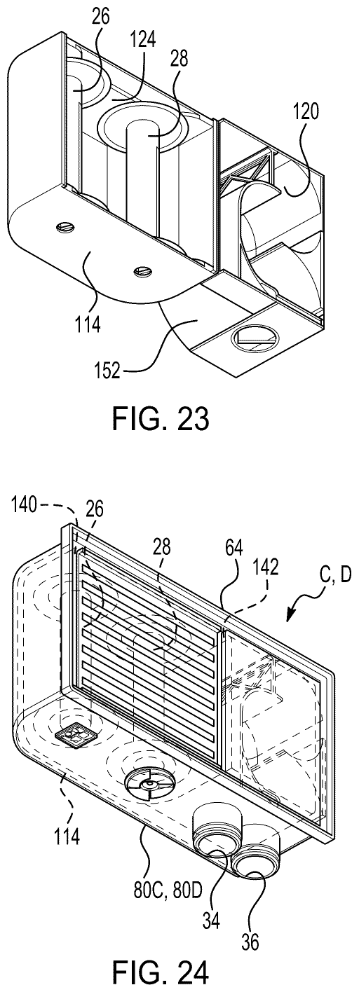

An example of the cartridges C, D is shown in FIG. 20 through FIG. 24. FIG. 20 depicts a perspective view of the exterior of the cartridge C, D with the cover 64 in place; FIG. 21 depicts a side view of the exterior of the cartridge C, D; FIG. 22 depicts a perspective view of an interior housing of the cartridge C, D with a cap 136 and a lid 138 in place; FIG. 23 depicts the inside of the interior housing of FIG. 22; and FIG. 24 depicts a perspective view of both the interior and the exterior of the cartridges C, D.

The housing 80C, 80D is shown in FIG. 20. In this example, the housing 80C, 80D, functions as an outer housing that defines a space for an interior housing 114, shown in FIG. 22 and in FIG. 23. The housing 80C, 80D also defines the inlet 34, the outlet 36, and an aperture for a drive/step motor coupler 116. The inlet 34 and the outlet 36 respectively attach, via gas conduits 96) to the gas inlet port 86′ and the outlet port 88′ of the main body 78′ (see FIG. 18 and FIG. 19). The drive/step motor coupler 116 operatively connects to the stepper motor 44C, which drives film 10 or 10″ advancement.

The cartridge C, D also includes the cover 64, which, in this example is transparent to the light hv used to release NO from the film 10 or 10″. The cover 64 may be placed on or secured to the housing 80C, 80D in order to create the chamber 24 where the film 10 or 10″ is exposed to light hv. As such, the chamber 24 is defined between the cap 136 and the cover 64. The attachment between the cover 64 and the housing 80C, 80D creates an airtight seal.

In the example shown in FIG. 20 and FIG. 21, the housing 80C, 80D also includes a designated region for holding a memory chip 118. The memory chip 118 may be electrically connected to the electronic circuitry 32 when the cartridge C, D, is inserted into the device 40′, and thus may receive and store data/information from the device 40″.

FIG. 22 depicts the interior housing 114 of the cartridge C, D with the cap 136 and lid 138 in place, and FIG. 23 depicts the interior housing 114 of the cartridge C, D with the cap 136 and lid 138 removed. The interior housing 114 may be formed of any of the materials set forth herein for the housing 80.

The frame of the interior housing 114 supports the cap 136 in a position adjacent to a region 124 of the interior housing 114 where the supply and pick-up reels 26, 28 are positioned, and also supports the lid 138 in a position adjacent to a region 120 where gases are removed from the cartridge C, D.

The cap 136 is formed of any material that is opaque to the light hv used to release NO from the film 10 or 10″. Examples of suitable materials for the cap 136 include polytetrafluoroethylene (PTFE), high density polyethylene (HDPE), acrylic, polycarbonate (PC), stainless steel, etc. The cap 136 is secured to the interior housing 114 so that the region 124, the supply and pick-up reels 26, 28 contained therein, and the film 10 or 10″ contained therein are covered by the cap 136. The securing mechanism may be an adhesive or a mechanical fastener. The cap 136 helps to keep NO from prematurely releasing from the cartridge C, D.

As shown in FIG. 22, an outlet slit 140 is defined between the top 144 of the interior housing 114 and one side 146 of the cap 136, and an inlet slit 142 is defined between the other side 148 of the cap 136 and an interior facing side 150 of the lid 138. While not shown in FIG. 22 through FIG. 24, the initially spooled film 10 or 10″ is wound on the supply reel 26 (see FIG. 23), is threaded through the outlet slit 140, positioned across the cap 136, and then threaded back through the inlet slit 142 where an end is attached to the pick-up reel 28. During operation, the film 10 or 10″ moves across the cap 136, within the chamber 24 defined between the cap 136 and the cover 64, in a direction from the outlet slit 140 toward the inlet slit 142. The light transparent cover 64 provides the window for the film 10, 10″ to be exposed to light hv.

Also during operation, the oxygen-containing gas OC (not shown in FIG. 20 through FIG. 24), is introduced into the inlet 34 and travels along panel 152 of the interior housing 114 and into a space that is defined between the exterior surface of the interior housing 114 and an interior surface of the housing 80C, 80D under the region 124 and along the top 144. The oxygen-containing gas OC then travels across the cap 136 and the chamber 24, where it picks up the released nitric oxide to form the output gas OG. The output gas OG is then transported into the region 120, which houses an NO₂ scrubber material. The NO₂ deficient output gas OG is then transported through the aperture 122″ of the interior housing 114 to the outlet 36. The output gas OG may be transported through a gas conduit 96 to the outlet port 88′ of the device 40″.

The lid 138 is secured to the interior housing 114 so that the region 120 is sealed. Sealing this region 120 enables the output gas OG transported therethrough to exit through the aperture 122″ alone. The securing mechanism may be an adhesive or a mechanical fastener. The lid 138 is formed of any of the opaque or light transparent materials disclosed herein.

As mentioned, the region 120 houses an NO₂ scrubber material. Any of the NO₂ scrubber materials described herein may be incorporated into the region 120. By sequestering NO₂ scrubber material in the region 120 between the chamber 24 and the outlet 36, NO₂ contained in the output gas OG is removed before the output gas OG is transported out of the cartridge C, D.

As shown in FIG. 22 and FIG. 23, the interior housing 114 includes apertures 122, 122′. The apertures 122, 122′ extend into the region 124. The aperture 122 enables a film spindle or rod to be inserted where it operatively connects to the supply reel 26 so that it rotates the supply reel 26 when advanced by the stepper motor 44C. The aperture 122′ provides access for the drive/step motor coupler 116 to operatively couple to the pick-up reel 28.

The exterior and the interior of the cartridge C, D is depicted in FIG. 24.

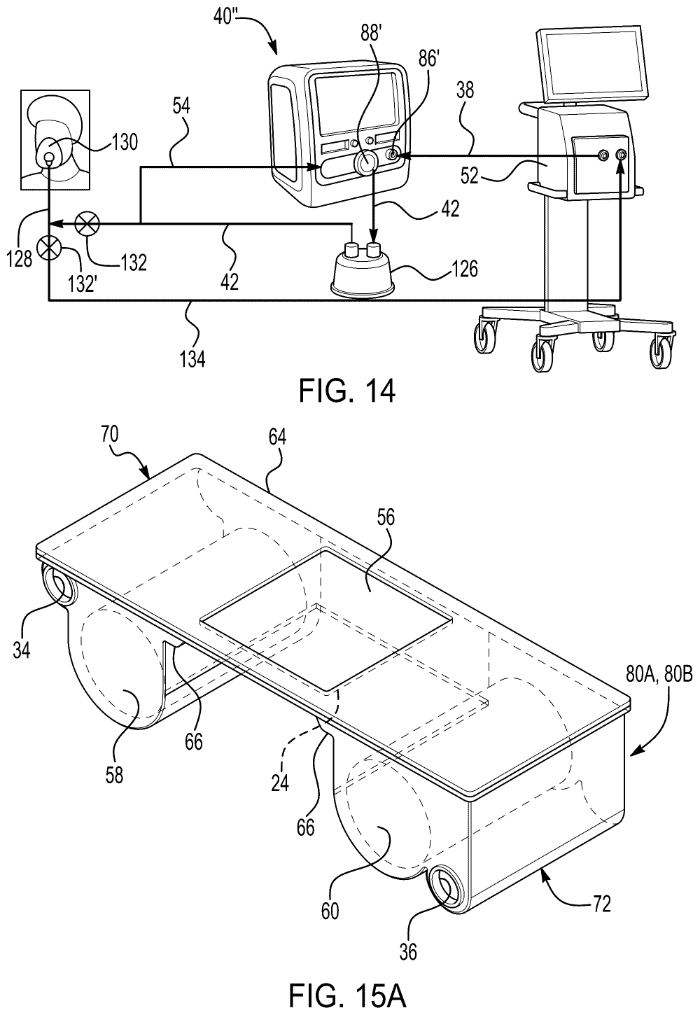

Referring back to FIG. 14, a schematic and perspective view of the portable nitric oxide releasing device (i.e., gas generating device 40″) is depicted in use in a patient care setting.

In this example, the gas source 52 is a ventilator that is operatively connected, via the inspiratory gas conduit 38 to the inlet port 86′ of the device 40″. The ventilator supplies the oxygen-containing gas OC to the device 40″ where it is mixed with the released nitric oxide via the film 10 or 10″ and method disclosed herein.

The output gas OG is transported through the conduit 42. In the example shown in FIG. 14, a humidifier 126 is positioned to receive the output gas OG from a portion of the conduit 42 and to transport a humidified output gas to the recipient. The humidifier 126 is included to humidify the output gas OG before it is introduced to the recipient so that his/her lungs do not dry out.

As shown, the sampling line 54 is positioned to send a portion of the humidified output gas back to the gas generating device 40″ for purposes of monitoring and feedback control, with use of sampling gas pump 102 (FIG. 19). The remainder of the output gas OG is delivered to the recipient via a delivery conduit 128 and an inhalation unit 130, such as a face mask, a nasal cannula, or some other suitable apparatus for delivering the output gas stream OG to the airways of the recipient.

As depicted in FIG. 14, respective inspiratory and expiratory ports 132, 132′ may be positioned in the conduit 42 and in an exhalation conduit 134 to direct gas out of the ventilator circuit upon recipient exhalation. For example, when the (humidified) output gas OG is being transported to the recipient, the gas goes through the inspiratory port 132 and then to the recipient through the delivery conduit 128. When the recipient breathes out, gas from the recipient travels through the delivery conduit 128, the expiratory port 132′, and into the exhalation conduit 134.

To further illustrate the present disclosure, examples are given herein. It is to be understood that these examples are provided for illustrative purposes and are not to be construed as limiting the scope of the present disclosure.

### NON-LIMITING WORKING EXAMPLES

### Example 1

0.047 g of TECOFLEX™ TPU was dissolved in 13 mL of a solvent mixture containing 10:3 cyclohexane:tetrahydrofuran to form a polymer solution. 1 g of GSNO was added to the polymer solution to form a slurry. The slurry was spray coated onto a TYVEK® film (having 0.45 μm pores, and dimensions of 7 cm×133 cm), and the solvent mixture was allowed to evaporate.

The NO releasing film secured to a spooling system similar to that shown in FIG. 3.

A target setpoint experiment was performed. In this experiment, the goal was to produce a target amount of NO (in parts per million (ppm)) at a given time by adjusting the light exposure and the speed of the belt of the spooling system. The light source was a light emitting diode (λ=395 nm), and a PID controller adjusted the voltage between 0 V (target NO=0 ppm) and 4.5 V (target NO=60 ppm). The stepper motor speed was initially set at 3 rpm, and then was stepped up 0.25 rpm for each interval going from the 0 ppm target interval to the 60 ppm target interval and then down 0.25 rpm for each interval going from the 60 ppm target interval to the 0 ppm target interval. Each interval ranged from 5 minutes to 8 minutes.

Air was transported through the spooling system housing and into the outlet conduit, which was in fluid communication with a sampling line containing nitric oxide (NO) and nitrogen dioxide (NO₂) sensors. The air in the housing of the spooling system picked up the generated NO and nitrogen dioxide NO₂, and then the mixed gaseous stream was directed into the outlet conduit. The flow rate of the air was 4.0 LPM (liters per minute), and 200 SCCM (standard cubic centimeters per minute) was directed into the sampling line and toward the sensors. More specifically, 4 LPM of air was directed into the chamber where it picked up the generated NO. The combination exited the chamber at 4 LPM. A sampling line pulled the 200 SCCM over the sensors for measurement.

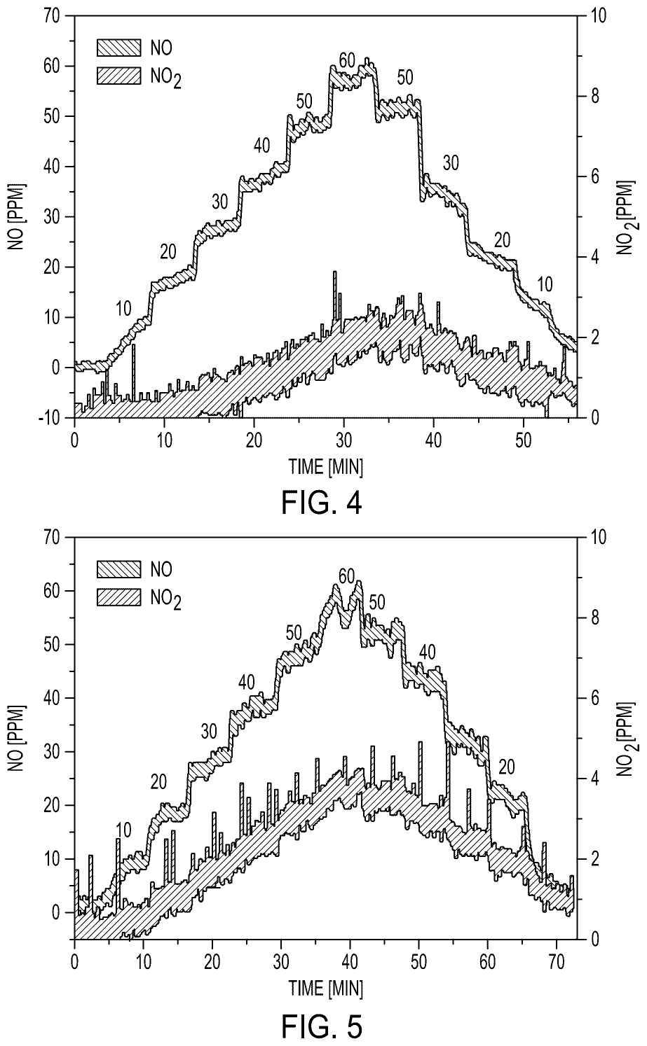

The results from this experiment are shown in FIG. 4. As depicted, the NO releasing film generated expected amounts of NO and minimal amounts of NO₂ at each of the intervals.

### Example 2

A slurry was generated as described in Example 1. The slurry was spray coated onto a polypropylene (PP) films (having 0.45 μm pores, and dimensions of 7 cm×133 cm), and the solvent mixture was allowed to evaporate.

The NO releasing film secured to a spooling system similar to that shown in FIG. 3.

A target setpoint experiment was performed. In this experiment, the goal was to produce a target amount of NO (in parts per million (ppm)) at a given time by adjusting the light exposure and the speed of the belt of the spooling system. The light source was a light emitting diode (λ=395 nm), and a PID controller adjusted the voltage between 0 V (target NO=0 ppm) and 4.5 V (target NO=60 ppm). The stepper motor speed was initially set at 3 rpm, and then was stepped up 0.25 rpm for each interval going from the 0 ppm target interval to the 60 ppm target interval and then down 0.25 rpm for each interval going from the 60 ppm target interval to the 0 ppm target interval. Each interval ranged from 5 minutes to 8 minutes.

Air was transported through the spooling system housing and into the outlet conduit, which was in fluid communication with a sampling line containing nitric oxide (NO) and nitrogen dioxide (NO₂) sensors. The air in the housing of the spooling system picked up the generated NO and nitrogen dioxide NO₂, and then the mixed gaseous stream was directed into the outlet conduit. The flow rate of the air was 4.0 LPM (liters per minute), and 200 SCCM (standard cubic centimeters per minute) was directed into the sampling line and toward the sensors.

The results from this experiment are shown in FIG. 5. As depicted, the NO releasing film generated expected amounts of NO and minimal amounts of NO₂ at each of the intervals. The slightly elevated NO₂ may be the result of NO being generated and not escaping from the polypropylene backing. The longer NO resonance time in the film may lead to more NO being converted to NO₂.

While not reproduced herein, additional data was collected with PP films with 1.0 μm pores and 5.0 μm pores. The larger pore size did not promote significantly better adhesion.

### Example 3

A slurry was generated as described in Example 1. The slurry was spray coated onto a synthetic polyamide film (having 0.45 μm pores, and dimensions of 7 cm×133 cm), and the solvent mixture was allowed to evaporate.

The NO releasing film secured to a spooling system similar to that shown in FIG. 3.

A target setpoint experiment was performed. In this experiment, the goal was to produce a target amount of NO (in parts per million (ppm)) at a given time by adjusting the light exposure and the speed of the belt of the spooling system. The light source was a light emitting diode (λ=395 nm), and a PID controller adjusted the voltage between 0 V (target NO=0 ppm) and 4.5 V (target NO=60 ppm). The stepper motor speed was initially set at 3 rpm, and then was stepped up 0.25 rpm for each interval going from the 0 ppm target interval to the 60 ppm target interval and then down 0.25 rpm for each interval going from the 60 ppm target interval to the 0 ppm target interval. Each interval ranged from 5 minutes to 8 minutes.

Air was transported through the spooling system housing and into the outlet conduit, which was in fluid communication with a sampling line containing nitric oxide (NO) and nitrogen dioxide (NO₂) sensors. The air in the housing of the spooling system picked up the generated NO and nitrogen dioxide NO₂, and then the mixed gaseous stream was directed into the outlet conduit. The flow rate of the air was 4.0 LPM (liters per minute), and 200 SCCM (standard cubic centimeters per minute) was directed into the sampling line and toward the sensors.

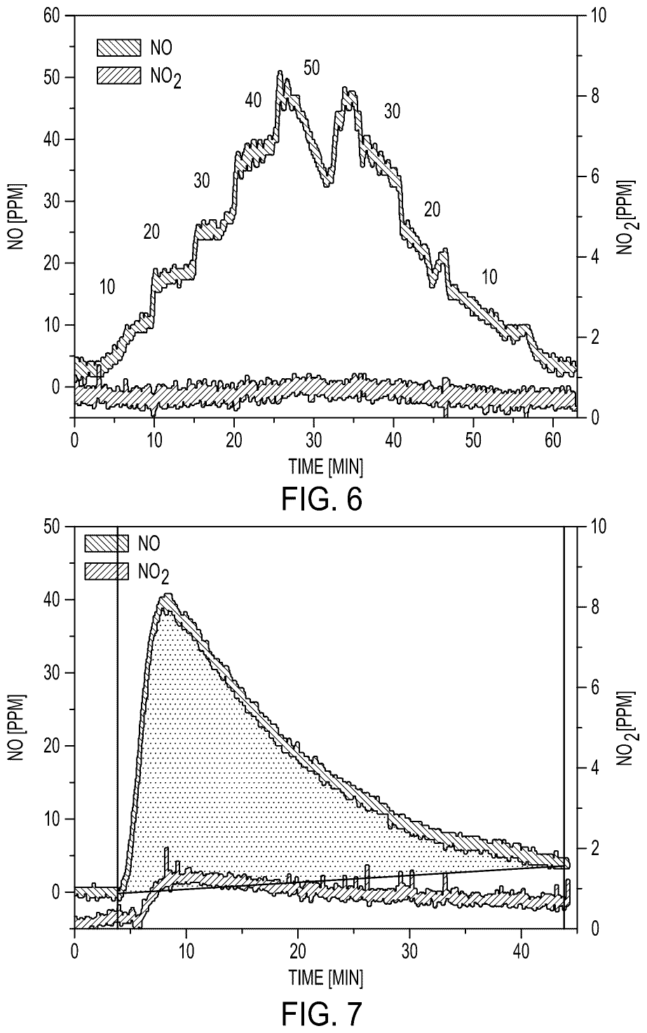

The results from this experiment are shown in FIG. 6. As depicted, the NO releasing film generated expected amounts of NO and minimal amounts of NO₂ at each of the intervals.

While not reproduced herein, additional data was collected with polyamide films with 1.0 μm pores and 5.0 μm pores. The larger pore size did not promote significantly better adhesion.

### Example 4

Seven slurries were generated as described in Example 1. Each slurry was spray coated onto a different 7 cm×7 cm substrate including: TYVEK® (having about 0.22 μm pores), polypropylene (having 0.45 μm pores), PELLON® (80/20 cotton/polyester blend), polyamide (having 0.45 μm pores), polytetrafluoroethylene (having 0.45 μm pores), glass fibers (having 0.45 μm pores), and polyvinylidene fluoride (having 0.45 μm pores). The solvent mixture was allowed to evaporate.

The NO releasing films were secured to a spooling system similar to that shown in FIG. 3.

A depletion experiment was performed with each of the NO releasing films. In this experiment, the goal was to determine how much NO (in parts per million (ppm)) could be generated from each of the NO releasing films. The light source was a light emitting diode (λ=395 nm), and the voltage maintained at 4.5 V. The stepper motor speed was set at 0 rpm, so that the film was stationary. The same portion of the film was exposed to the light for the entire experiment.

Air was transported through the spooling system housing and into the outlet conduit, which was in fluid communication with a sampling line containing nitric oxide (NO) and nitrogen dioxide (NO₂) sensors. The air in the housing of the spooling system picked up the generated NO and nitrogen dioxide NO₂, and then the mixed gaseous stream was directed into the outlet conduit. The flow rate of the air was 4.0 LPM (liters per minute), and 200 SCCM (standard cubic centimeters per minute) was directed into the sampling line and toward the sensors.

For each film, light exposure was performed for about 45 minutes. The results for the NO releasing film including the TYVEK® substrate are shown in FIG. 6. As depicted, the NO releasing film generated high levels of NO after about 5 minutes of light exposure and then the amount decreased as times passed. Minimal amounts of NO₂ were generated. The raw data results for the other NO releasing films are not reproduced herein.

The area under the NO curve was determined for each of the NO releasing film, and these results are shown in Table 2.

TABLE 2

| Carrier Material of NO | Total Integrated NO |
|---|---|
| releasing film | Release (ppm) |
| TYVEK ® | 585 |
| polypropylene | 421 |
| PELLON ® | 409 |
| polyamide | 401 |
| polytetrafluoroethylene | 307 |
| glass fibers | 251 |
| polyvinylidene fluoride | 235 |

Each of the first five NO releasing films generated desirable amounts of NO, while the last two NO releasing films (made with glass fibers and PVDF) generated mediocre amounts of NO. It was noted that some of the substrate materials, including glass fibers and polyvinylidene fluoride, may be less desirable as the NO releasing film could be rubbed off of these substrates.

### Example 5

A slurry was generated as described in Example 1. The slurry was spray coated onto four different 7 cm×7 cm TYVEK® substrates, and the solvent was allowed to evaporate from each.

The NO releasing films were held stationary for this experiment, and each was exposed to a different UV-A wavelength. The light sources were light emitting diodes (λ=365 nm, λ=385 nm, λ=395 nm, and λ=405 nm), and light exposure was continued for about 15 minutes.

Air was transported through the system housing the stationary films and into an outlet conduit, which was in fluid communication with a sampling line containing nitric oxide (NO) sensors. The air in the housing of the system picked up the generated NO from the film surface, and then the gaseous stream was directed into the outlet conduit. The flow rate of the air was 4.0 LPM (liters per minute), and 200 SCCM (standard cubic centimeters per minute) of the total airflow was directed into the sampling line and toward the NO sensors.

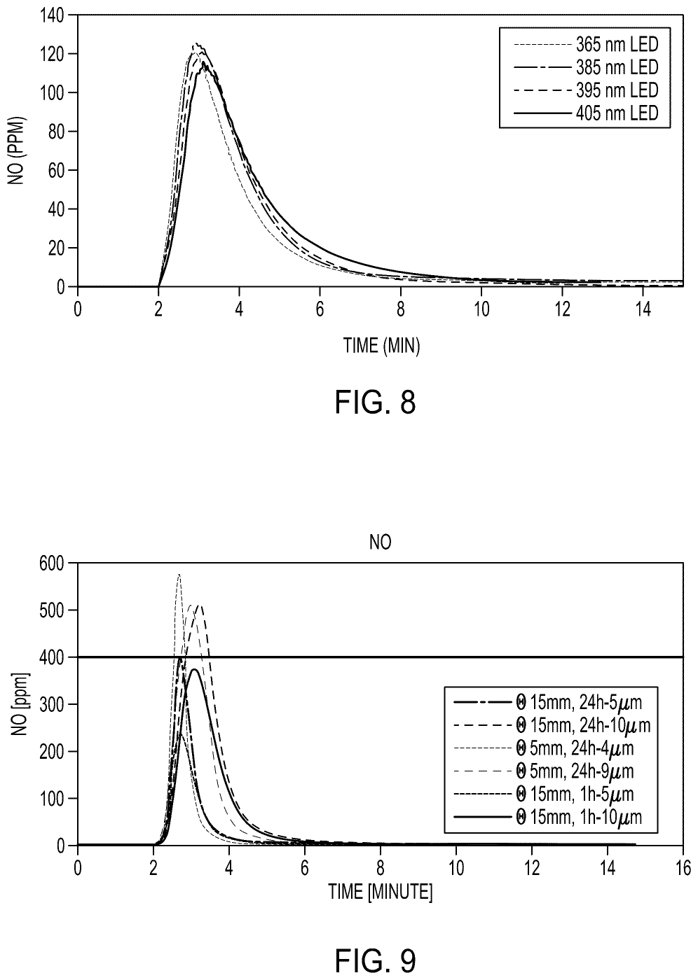

The results from this experiment are shown in FIG. 8. As depicted, each of the light sources performed similar and a similar amount of NO was generated from each of the films.

### Example 6

GSNO was exposed to ball milling to generate differently sizes particles. The ball mill size, the ball mill exposure time, and resulting GSNO particle size varied from one sample to the next. A total of six different samples of GSNO were generated.

Respective slurries were generated with the GSNO samples and ethanol as the solvent. The slurries were respectively micro gravure printed onto PET substrates that had been coated with polyvinyl butyral and dried. The solvent was allowed to evaporate from each at a drying temperature of 60° C.

The NO releasing films were held stationary for this experiment, and each was exposed to a UV-A wavelength of 395 nm, and light exposure was continued for about 15 minutes.

Air was transported through the system housing the stationary films and into an outlet conduit, which was in fluid communication with a sampling line containing nitric oxide (NO) sensors. The air in the housing of the system picked up the generated NO from the film surface, and then the gaseous stream was directed into the outlet conduit. The flow rate of the air was 4.0 LPM (liters per minute), and 200 SCCM (standard cubic centimeters per minute) of the total airflow was directed into the sampling line and toward the NO sensors.

The results from this experiment are shown in FIG. 9. Several of the samples reached NO peaks over 400 ppm. The ϕ mm measurement (e.g., ϕ15 mm) is the diameter of the ball media used to grind and pre-process the GSNO. The time measurement (e.g., 24 h) associated with each data series was the time of ball milling (GSNO with these balls to break up and reduce the GSNO particle size). The μm measurement (e.g., 5 μm) associated with each data set refers to the D50 particle size of the GSNO particles themselves. As depicted, longer ball milling times (up to 24 h) generated NO donor particles that lead to the creation of a more stable, more reproducible NO donor film. In turn, a more stable film leads to more efficient and reproducible NO release.

### Example 7

78.3 g of polystyrene-block-polyisoprene-block-polystyrene was dissolved in 13.4 g ethanol and 120.7 g n-butylacetate to form a polymer solution. The polymer solution was combined with 600.0 g of GSNO, 75.6 g of a 50 wt % solution of Lubrizol Solsperse™ M-389 dispersant (37.8 g dispersant+37.8 g n-butylacetate), and an additional 52.5 g ethanol and 384.4 g n-butylacetate was added to form a suspension. This suspension was ball-milled for 1 hour to generate a desirable particle size distribution and slurry. The slurry was then applied onto a PET film via slot-die coating and the solvent residue was allowed to evaporate.

The resulting NO releasing film was secured to a stationary test bed with an illumination window similar to that of the cartridge window shown in FIG. 3.

A depletion experiment was performed. In this experiment, the goal was to evaluate how much NO (in parts per million (ppm)) could be generated from the NO releasing film that had been coated by slot-die coating. The light source was a 6×4 array of light emitting diodes (λ=395 nm), and the voltage was maintained at 13.3 V. The film was kept stationary such that the same portion of the film was exposed to the light for the entire experiment.

Air was transported through the housing of the stationary test bed and into an outlet conduit, which was in fluid communication with a sampling line containing nitric oxide (NO) and nitrogen dioxide (NO₂) sensors. The air in the housing of the stationary test bed picked up the generated NO and nitrogen dioxide NO₂, and then the mixed gaseous stream was directed into the outlet conduit. The flow rate of the air was 10.0 LPM (liters per minute), and 200 SCCM (standard cubic centimeters per minute) was directed into the sampling line and toward the sensors.

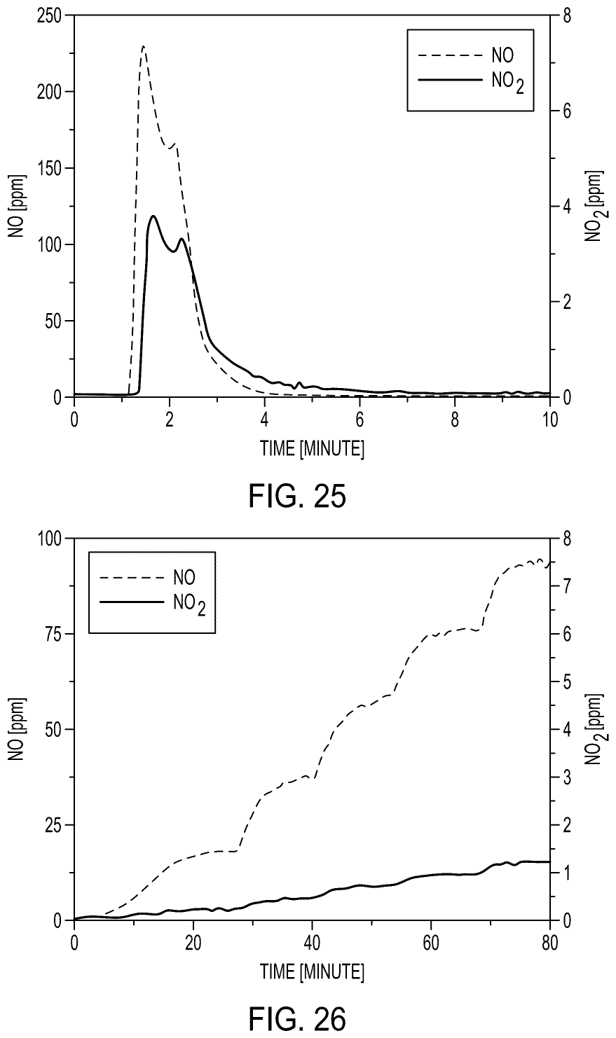

Light exposure was performed for about 15 minutes. The results for the NO film are shown in FIG. 25. As depicted, the NO releasing film generated high levels of NO after about 30 seconds of light exposure and then the amount decreased as time passed. Up to 3.8 ppm of NO₂ was generated shortly after up to 226 PPM of NO was generated.

### Example 8

53.1 g of polystyrene-block-polyisoprene-block-polystyrene was dissolved in 7.7 g ethanol and 69.6 g n-butyl acetate to form a polymer solution. The polymer solution was combined with 150.0 g GSNO and an additional 51.5 g ethanol and 451.3 g n-butyl acetate to form a suspension. This suspension was ball-milled for 6 hours to generate a desirable particle size distribution and slurry. The slurry was then applied onto a PET film via gravure and the solvent residue was allowed to evaporate.

The NO releasing film was secured to a spooling system similar to that shown in FIG. 3.

A target setpoint experiment was performed. In this experiment, the goal was to produce a target amount of NO (in parts per million (ppm)) at a given time by adjusting the speed of the belt of the spooling system at a constant LED intensity. The light source was a 2×4 light emitting diode array (Δ=395 nm). The stepper motor speed was initially set at a step rate of 1, and was stepwise ramped to step rates of 2, 3, 4, and 6 after sustained levels of NO and NO₂ were generated (around 7 to 20 minutes per step).

Air was transported through the spooling system housing and into the outlet conduit, which was in fluid communication with a sampling line containing nitric oxide (NO) and nitrogen dioxide (NO₂) sensors. The air in the housing of the spooling system picked up the generated NO and nitrogen dioxide NO₂, and then the mixed gaseous stream was directed into the outlet conduit. The flow rate of the air was 10.0 LPM (liters per minute), and 200 SCCM (standard cubic centimeters per minute) was directed into the sampling line and toward the sensors.

The results from this experiment are shown in FIG. 26. As depicted, the NO releasing film generated expected amounts of NO (left Y axis) as a function of film advancement rate and generated minimal amounts of NO₂ (right Y axis) at each of the intervals.

### ADDITIONAL NOTES

It is to be understood that the ranges provided herein include the stated range and any value or sub-range within the stated range, as if the value(s) or sub-range(s) within the stated range were explicitly recited. For example, a range from about 250 nm to about 600 nm should be interpreted to include not only the explicitly recited limits of from about 250 nm to about 600 nm, but also to include individual values, such as about 375 nm, about 520.5 nm, 450 nm, 599 nm, etc., and sub-ranges, such as from about 395 nm to about 595 nm, etc. Furthermore, when “about” is utilized to describe a value, this is meant to encompass minor variations (up to +/−10%) from the stated value.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

In describing and claiming the examples disclosed herein, the singular forms “a”, “an”, and “the” include plural referents unless the context clearly dictates otherwise.

While several examples have been described in detail, it is to be understood that the disclosed examples may be modified. Therefore, the foregoing description is to be considered non-limiting.

What is claimed is:

1. A nitric oxide (NO) releasing film, comprising:
a polymer matrix selected from the group consisting of a non-UV curable polyurethane, polyvinyl butyral, polystyrene, copolymers of styrene, block copolymers of styrene, poly(ethersulfone), polyvinylpyrrolidone, polyvinyl acetate, poly(ethylene-co-vinylacetate), and combinations thereof; and
solid, light sensitive NO donor particles distributed throughout the polymer matrix, wherein a volume-weighted mean diameter of the solid, light sensitive NO donor particles is 50 μm or less.

2. The NO releasing film as defined in claim 1, wherein the solid, light sensitive NO donor particles are a light sensitive S-nitrosothiol selected from the group consisting of S-nitroso-N-acetyl-penicillamine (SNAP), S-nitrosoglutathione (GSNO), S-nitroso-N-acetylcysteine (SNAC), S-nitroso-3-β-mercaptopropionic acid (SN3-MPA), and combinations thereof.

3. The NO releasing film as defined in claim 1, wherein the volume-weighted mean diameter ranges from about 0.1 μm to 50 μm.

4. The NO releasing film as defined in claim 1, wherein a weight ratio of the solid, light sensitive NO donor particles to the polymer matrix ranges from about 0.1:1 to about 50:1.

5. The NO releasing film as defined in claim 1, further comprising an additive selected from the group consisting of NO₂ scrubber particles, a radical stabilizer, a dispersant, an anti-skinning additive, and combinations thereof.

6. The NO releasing film as defined in claim 5, wherein a mole ratio of the NO₂ scrubber particles to the NO donor particles ranges from about 0.02:1 up to 1:1.

7. The NO releasing film as defined in claim 5, wherein a mole ratio of the radical stabilizer to the NO donor particles ranges from about 0.02:1 up to 1:1.

8. The NO releasing film as defined in claim 1, wherein:
the polymer matrix and the solid, light sensitive NO donor particles distributed throughout the polymer matrix form an NO donor film; and
the NO releasing film further comprises a substrate attached to the NO donor film.

9. The NO releasing film as defined in claim 8, further comprising an NO permeable and light transparent film positioned on the NO donor film.

10. The NO releasing film as defined in claim 8, further comprising a base binding layer positioned between the substrate and the NO donor film.

11. The NO releasing film as defined in claim 10, wherein the base binding layer is an adhesive or a primer layer that promotes attachment of the NO donor film to the substrate.

12. The NO releasing film as defined in claim 8, wherein the substrate is a porous or non-porous material formed of a polymer that exhibits low O₂ permeability/solubility.

13. A method, comprising:
dissolving a polymer matrix selected from the group consisting of a non-UV curable polyurethane, polyvinyl butyral, polystyrene, copolymers of styrene, block copolymers of styrene, poly(ethersulfone), polyvinylpyrrolidone, polyvinyl acetate, poly(ethylene-co-vinylacetate), and combinations thereof, thereby producing a polymer solution;
mixing solid, light sensitive NO donor particles into the polymer solution thereby producing a coating slurry, wherein a volume-weighted mean diameter of the solid, light sensitive NO donor particles is 50 μm or less;
depositing the coating slurry on a support selected from the group consisting of a temporary substrate and a substrate that is impermeable to the solvent mixture; and
evaporating the solvent mixture, thereby producing an NO donor film on the support.

14. The method as defined in claim 13, wherein:
the support is the substrate that is impermeable to the solvent mixture; and
prior to depositing the slurry on the substrate, the method further comprises forming a base binding layer on the substrate, and wherein the slurry is deposited on the base binding layer.

15. The method as defined in claim 13, wherein:
the support is the temporary substrate; and
the slurry is cast on the temporary substrate.

16. The method as defined in claim 15, further comprising removing the NO donor film from the temporary substrate.

17. The method as defined in claim 13, further comprising adding an additive to the polymer solution, wherein the additive is selected from the group consisting of NO₂ scrubber particles, a radical stabilizer, a dispersant, an anti-skinning additive, and combinations thereof.

18. A nitric oxide releasing system, comprising:
a chamber;
a spooling system including a supply reel and i) a motor-controlled pick-up reel or ii) a transfer reel and a waste apparatus;
a nitric oxide (NO) releasing film wound on the supply reel and having an end attached to the motor-controlled pick-up reel or the waste apparatus, the NO releasing film including:
a substrate;
an NO donor film attached to the substrate, the NO donor film including:
a polymer matrix selected from the group consisting of a non-UV curable polyurethane, polyvinyl butyral, polystyrene, copolymers of styrene, block copolymers of styrene, poly(ethersulfone), polyvinylpyrrolidone, polyvinyl acetate, poly(ethylene-co-vinylacetate), and combinations thereof; and
solid, light sensitive NO donor particles distributed throughout the polymer matrix, wherein a volume-weighted mean diameter of the solid, light sensitive NO donor particles is 50 μm or less; and
an NO permeable and light transparent film positioned on the NO donor film; and
a light source operatively positioned to selectively expose a spooled portion of the NO releasing film to light to release NO gas.

19. A gas release and delivery device, comprising:
a nitric oxide (NO) releasing system, including:
a chamber;
a spooling system including a supply reel and i) a motor-controlled pick-up reel or ii) a transfer reel and a waste apparatus;
a nitric oxide (NO) releasing film wound on the supply reel and having an end attached to the motor-controlled pick-up reel or the waste apparatus, the NO releasing film including:
a substrate;
an NO donor film attached to the substrate, the NO donor film including:
a polymer matrix selected from the group consisting of a non-UV curable polyurethane, polyvinyl butyral, polystyrene, copolymers of styrene, block copolymers of styrene, poly(ethersulfone), polyvinylpyrrolidone, polyvinyl acetate, poly(ethylene-co-vinylacetate), and combinations thereof; and
solid, light sensitive NO donor particles distributed throughout the polymer matrix, wherein a volume-weighted mean diameter of the solid, light sensitive NO donor particles is 50 μm or less; and
an NO permeable and light transparent film positioned on the NO donor film; and
a light source operatively positioned to selectively expose a portion of the NO releasing film to light to release NO gas;
an inspiratory gas conduit operatively connected to the chamber to introduce an oxygen-containing gas and form an output gas including the NO gas; and
an outlet conduit to transport a stream of the output gas from the NO releasing system.

20. The gas release and delivery device as defined in claim 19, wherein the NO donor film further comprises an additive selected from the group consisting of NO₂ scrubber particles, a radical stabilizer, a dispersant, an anti-skinning additive, and combinations thereof.

21. The gas release and delivery device as defined in claim 19, further comprising:
at least two sets of sensors to monitor NO, NO₂ and O₂; and
a feedback and monitoring system to receive data from the at least two sets of sensors.

22. The gas release and delivery device as defined in claim 19, further comprising:
a main body including two cartridge slots that are to removably receive respective NO releasing systems;
an oxygen-containing source operatively connected to an inlet port of the main body; and
an automated switching valve that, when in operation, directs flow from the oxygen-containing source to one of the respective NO releasing systems to provide continuous NO delivery.

23. The gas release and delivery device as defined in claim 22, wherein the automated switching valve is configured to:
provide some flow to one of the respective NO releasing systems when switching flow to an other of the respective NO releasing systems to maintain uninterrupted flow through the device; and
shut off flow through either of the NO releasing systems to isolate the one or the other of the respective NO releasing systems.

24. The gas release and delivery device defined in claim 19, further comprising an automated calibration system operatively connected to the NO releasing system.
